(19) United States
(12) Patent Application Publication
(10) Pub. No.: US 2007/0037148 A1
(43) Pub. Date: Feb. 15, 2007

(54) Compositions and methods for the treatmetn of natural killer cell related diseases

(76) Inventors: Sherman Fong, Alameda, CA (US); Kathryn Dennis, Los Gatos, CA (US); Hilary Clark, San Francisco, CA (US); Henry Chiu, San Francisco, CA (US); Jill Schoenfeld, Ashland, OR (US); P. Mickey Williams, Half Moon Bay, CA (US); William I. Wood, Cupertino, CA (US); Thomas D. Wu, San Francisco, CA (US)

Correspondence Address: GENENTECH, INC., 1 DNA WAY, SOUTH SAN FRANCISCO, CA, 94080, US

(21) Appl. No.: 10/533,416

(22) PCT Filed: Nov. 6, 2003
(86) PCT No.: PCT/US03/35268

§ 371 Date: Apr. 17, 2006

Related U.S. Application Data
(60) Provisional application No. 60/193,767, filed on Mar. 31, 2000.

(51) Int. Cl. C12Q 1/68; C12M 1/34

(52) U.S. Cl. 435/6; 435/287.2; 427/2.11

(57) ABSTRACT

Compositions and methods for improving detection sensitivity in nucleic acid microarray analysis are disclosed, including methods of purifying nucleic acids, methods of synthesizing fluorescent DNA probes, methods of hybridization, and methods of activating a substrate for target molecule attachment are disclosed.

### FIELD OF THE INVENTION

This invention relates to compositions and methods for improved analysis of gene expression, genetic polymorphism or gene mutation using nucleic acid microarrays for genetic research and diagnostic applications.

### BACKGROUND

Nucleic acid microarrays, often containing thousands of gene sequences, are useful for identifying differential gene expression in diseased tissue relative to normal tissue of the same type, for example. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes that have potential to be expressed in certain disease states may be arrayed on a solid support. Hybridization of a labeled probe with a particular array member indicates that the sample from which the probe was derived expresses that gene. Differential gene expression analysis of disease tissue can provide valuable information. For example, if hybridization of a probe from a test (disease tissue) sample is greater than hybridization of a probe from a control (normal tissue) sample, the gene or genes expressed in the diseased tissue may be a significant diagnostic indicator of a potential drug target.

Detection sensitivity is a limiting factor for effectively analyzing test versus control samples such that gene expression, a genetic polymorphism, or a gene mutation associated with the disease may be recognized. For the study of human genes using DNA microarrays, successful analysis of many disease states requires sensitive detection to work with limiting sample quantities.

### SUMMARY

The present invention relates to the discovery that detection of genetic differences, such as gene expression, genetic polymorphism, or gene mutation, in diseased tissue relative to normal tissue, between tissues at different developmental states, between individuals, and like comparisons, is improved by the compositions and methods disclosed herein. The compositions and methods are useful for quantifying the relative amount or a component of a cell, where the component is a nucleic acid (including a polynucleotide DNA or RNA), a polypeptide, a protein, an antibody, and the like, by determining the amount of a particular complex formed between the component (or its equivalent) and a target molecule on a support surface. For example, where the component is a mixture of polynucleotides from a first biological sample and a second biological sample, and the target molecule is a known or knowable nucleic acid sequence, the complexes are a hybridization complex between the target molecule and the first and/or second polynucleotides. The component is preferably labeled as a detectable probe such that the complexes are distinguishable one from the other and the relative amounts of the complexes may be determined as a measure of the amount of the component present in the First biological sample relative to the second biological sample.

In one aspect, the invention involves a microarray. The microarray of the invention comprises target molecules arrayed on a solid support substrate in distinct spots that are at known, knowable or determinable locations within the array on the support substrate. A spot refers to a region of target molecule attached to the support substrate as a result of contacting a solution comprising target molecule with the substrate. Preferably, each spot is sufficiently separated from each other spot on the substrate such that they are distinguishable from each other during detection of complex formation. The microarray of the invention comprises at least one spot/cm², 20 spots/cm², 50 spots/cm², 100 spot/cm², and greater densities, including at least 300 spots/cm², 1000 spots/cm², 3000 spots/cm², 10,000 spots/cm², 30,000 spots/cm², 100,000 spots/cm , 300,000 spots/cm² or more as the available technology allows. Preferably, the microarray of the invention comprises at least 2000 spots/cm² to 25,000 spots/cm².

In an embodiment, the invention involves a microarray of biopolymers on a solid support substrate, wherein the substrate is silanized and the silanization occurs with a silanizing agent in toluene as the solvent and in the absence of acetone or an alcohol (such as methanol, ethanol, propanol, butanol, or the like). In a preferred embodiment the silanizing agent is an organosilane and the solvent toluene is substantially dry, wherein the drying is by standard techniques known in the art. The organosilane may be any organosilane comprising an alkyl or aryl linker between the silicon atom and a reactive functionality capable of forming a covalent bond with a functionality on the biopolymer or on another linker molecule useful in the invention. Preferably, the alkyl or aryl linker of the organosilane is from one to 20 carbon atoms in length, preferably from 1 to 15, and most preferably from 2 to 6 carbon atoms, inclusive. In a related embodiment, the organosilane comprises a functionality that is capable of covalently attaching to the biopolymer directly or indirectly through another linker molecule. The functionality on the organosilane may be, for example, an epoxide, a halide, a thiol, or a primary amine (see, for example, U.S. Pat. No. 6,048,695; U.S. Pat. No. 5,760,130; WO 01/06011; WO 00/70088, published Nov. 23, 2000). A useful organosilane for practicing the invention is, for example, 3-aminopropyl triethoxysilane (APS) (see, for example, WO 01/06011; WO 60/40593; U.S. Pat. No. 5,760,130; and Weiler et al., Nucleic Acids Research 25(14):2792-2799 (1997)). According to this embodiment, the invention involves a microarray comprising a biopolymer covalently attached to a substrate wherein the substrate is silanized with a silanizing agent, and wherein the substrate is reacted with the silanizing agent in toluene in the absence of acetone or an alcohol, such as methanol, for example.

In another embodiment, the invention involves a microarray wherein the covalent attachment of the biopolymer to the substrate is indirect, such as, for example, through a linker molecule. Thus, according to this embodiment, the invention involves a microarray comprising a biopolymer attached to a silanized substrate, wherein the microarray comprises a linker molecule between a substrate-attached silane and the biopolymer. According to a related embodiment, the microarray comprises a biopolymer, a silanizing agent, a multifunctional linker reagent, and a substrate, wherein the biopolymer is attached to the multifunctional linker reagent, the multifunctional linker reagent is attached to the biopolymer and the silanizing agent, and the silanizing agent is attached to the substrate by a reaction in toluene in the absence of acetone or alcohol. Preferably, the attachment between the biopolymer and the multifunctional linker reagent is covalent. Preferably, the attachment between the multifunctional linker reagent and the silanizing agent is covalent. Preferably, the substrate is glass and the reaction between the silanizing agent and substrate forms a covalent bond. In a preferred embodiment, the attachments, whether covalent or non-covalent, are sufficiently strong such that the biopolymer remains in its original spot within the array during complex formation, washing steps, and detection steps of microarray analysis. For an example of non-covalent attachment of nucleic acids and oligonucleotide probes in array hybridization reactions, see, for example WO 01/06011.

According to a related embodiment, the microarray of the invention is prepared by a method comprising silanizing a substrate, such as glass, with a silanizing agent in toluene in the absence of acetone or alcohol, followed by reacting a reactive functionality of the substrate-attached silanizing agent with a biopolymer to generate a biopolymer attached to a substrate. Preferably, the biopolymer is unmodified prior to reacting with the substrate-attached silanizing agent. Alternatively, the biopolymer is modified with a reactive functionality that reacts with a functionality of the substrate-attached silanizing agent.

In a related embodiment, the microarray of the invention is prepared by a method comprising silanizing a substrate, such as glass, with a silanizing agent in toluene in the absence of acetone or an alcohol, followed by reacting the substrate-attached silanizing agent with a multifunctional linker reagent at one of its functionalities, followed by reacting another of the functionalities with a biopolymer. Preferably, the biopolymer is unmodified prior to reacting with the multifunctional linker reagent of the substrate-silanizing agent-multifunctional linker reagent linkage. Optionally, the biopolymer is modified with a reactive functionality that reacts with a reactive functionality on the multifunctional linker reagent of the substrate-silanizing agent-multifunctional linker reagent linkage. The biopolymer may be modified by any procedure appropriate for the biopolymer of interest. For example, where the biopolymer is a polynucleotide, a reactive functionality may be introduced into the polynucleotide during its synthesis or after it is synthesized. According to a non-limiting example disclosed herein, a primary amine is a reactive functionality introduced into the polynucleotide as a derivatized nucleic acid primer. Preferably, the multifunctional linker reagent comprises two or more pendent chemically reactive groups (functionalities) adapted to form a covalent bond with a corresponding functional group on a substrate surface and adapted to form a covalent bond with a corresponding functional group on a target molecule.

According to a related embodiment, a substrate surface of a microarray slide is derivatized with a silanizing agent and, optionally, with the multifunctional linker reagent to activate the microarray slide for immobilizing the target molecule, wherein the activating comprises (1) silanizing the surface with an organosilane in toluene, preferably in the absence of acetone or an alcohol (such as methanol, for example), wherein the organosilane comprises a functionality reactive with the multifunctional linker reagent, and wherein the activating further comprises immobilizing the multifunctional linker reagent on the silanized surface by covalently reacting a first pendent reactive group of the multi functional linker reagent with the reactive functionality of the organosilane; (2) providing a solution comprising a target molecule having one or more functional groups reactive with a second pendent reactive group of the immobilized multi functional linker reagent; and (3) attaching the target molecule to the substrate surface by contacting the target molecule with the activated substrate surface and allowing a functional group of the target molecule to form a covalent bond with the second pendent reactive group of the immobilized multifunctional linker reagent.

In an embodiment of the invention, the target molecule of the microarray is a nucleic acid, such as a polynucleotide of RNA, single stranded or double stranded DNA, a synthetic oligonucleotide, a peptide nucleic acid (PNA) in which the backbone is a polypeptide backbone rather than a ribose or deoxyribose backbone, a polypeptide, a protein, an antibody, a receptor, a ligand, or like molecule that is detectable by its ability to form a complex with another molecule, a detectable complexing agent. The polynucleotide may be from 5 nucleotides in length to and including 10 kb in length. Preferably, the polynucleotide is from approximately 100 bp to 5 kb, more preferably from 0.3 kb to 3 kb, and even more preferably from approximately 0.5 kb to 2 kb. In an embodiment in which the target polynucleotide is PCR amplified double stranded DNA, the length is preferably from 0.5 to approximately 2 kb. In an embodiment in which the target polynucleotide is a chemically synthesized oligonucleotide, the length is preferably from approximately 50-1000 nucleotides, 50-500 nucleotides, 50-200 nucleotides, 50-100 nucleotides.

In another embodiment, the invention involves a microarray of the invention wherein the attached target molecule is a modified polynucleotide and the modification is addition of an amine to the native polymer. Preferably the amine is a primary amine and is preferably at the 5′ end of the polynucleotide, but may be incorporated elsewhere, depending on the constraints of polynucleotide preparation or the needs of the microarray assay. Where a reactive group, such as a primary amine, is preferred to be at the 5′ end of a polynucleotide, the primary amine may be part of a primer that is enzymatically extended to produce the primary amine-modified polynucleotide.

In still another embodiment, the substrate surface of the microarray of the invention comprises material selected from the group consisting of polymeric materials, glasses, ceramics, natural fibers, nylon and nitrocellulose membranes, gels, silicons, metals, and composites thereof. Preferably the substrate is glass, more preferably a glass slide. Preferably the microarray substrate comprises at least one flat surface comprising at least one of these materials. Optionally, the substrate is in a form of threads, sheets, films, gels, membranes, beads, plates, and like structures.

In another embodiment, the microarray of the invention is prepared by contacting the target molecule with an activated substrate by a technique from the group consisting of printing, capillary device contact printing, microfluidic channel printing, deposition on a mask, and electrochemical-based printing, wherein the contacting creates a discrete target molecule-containing spot on the substrate (See, for example, U.S. Pat. No. 5,700,637, U.S. Pat. No. 5,445,934, and U.S. Pat. No. 5,807,522 for particular methods of array formation, or Cheung, V. G. et al., Nature Genetics 21 (Suppl): 15-19 (1999) for a discussion of array fabrication). It is understood that various additional contacting techniques are well known in the art or may be developed for depositing a target molecule to a solid support. Preferably, a technique is chosen that is accurate, efficient, and economical for the user. In preferred embodiments where the target molecule is a modified or unmodified polynucleotide, the target polynucleotide is contacted with the substrate in a solution, wherein the concentration of target polynucleotide in the solution is preferably the range of 0.1 μg/μl lo and including 3 μg/μl. The pH of the solution is in the range from approximately pH 6-10, preferably approximately pH 6.5-9.7, more preferably approximately pH 7-9.4. Preferably, the target polynucleotide solution further comprises 500 mM sodium chloride, 100 mM sodium borate, pH 9.3. Preferably, once the target biopolymer is contacted with the substrate under conditions according to the invention the reaction is rapid, preferably 1 hour or less, 30 minutes or less, 10 minutes or less, or live minutes or less. It was discovered as part of the invention that allowing more time for the target polynucleotide to react with the activated slide improves detection sensitivity. For example, where the target polynucleotide is a double stranded or single stranded cDNA comprising a primary amine functionality and the activated slides are prepared according to the present invention, the spotted slides are allowed to remain at ambient temperature and humidity for from 1-24 hours, preferably about 5-18 hours, more preferably about 10-16 hours, and even more preferably about 12-14 hours before washing the slides to remove unreacted target molecule and other spotting solution components in preparation for hybridization and detection procedures.

According to the embodiment, the invention also involves blocking unreacted activating functionalities on the surface (e.g. unreacted silanizing agent and/or unreacted multifunctional linker linker reagent). Blocking reactions useful in the invention include washing the slides with water.

In another aspect, the invention involves an activated microarray slide, wherein the term “slide” refers to a solid support comprising at least one substantially flat surface and the term “activated” refers to the presence of reactive groups on the slide capable of reacting with a modified or unmodified target biopolymer according to the invention to cause the target biopolymer to be immobilized on the surface, such as by covalent or non-covalent attachment. Preferably, the activated slide comprises a silanized surface wherein the silanization occurred in toluene in the absence of acetone or an alcohol, such as methanol, for example.

In a preferred embodiment, the activated slide further comprises a multifunctional linker reagent that is capable of linking the surface-attached silanizing agent to the target biopolymer, thereby being capable of immobilizing the target biopolymer on the microarray slide. Preferably, the multifunctional linker reagent reacts first with a reactive functionality on the silanizing agent leaving at least one pendent reactive group on the multifunctional linker reagent capable of forming an attachment with a functional group of the target molecule, wherein the attachment is non-covalent or covalent as long as the target molecule remains attached at its original location in the array. In a preferred embodiment, the surface comprises glass pretreated by silanizing in toluene in the absence or acetone or in alcohol with an organosilane comprising at least one reactive functionality that is reactive with at least one pendent reactive group of the multifunctional linker reagent for immobilizing the multifunctional linker reagent.

In a preferred embodiment the target molecule is a polynucleotide and the functional group of the target molecule is a hydroxy group, an epoxide, or an amine. Where the functional group on the target polynucleotide is an amine, it is preferably a primary amine. Optionally, the primary amine is preferably at the 5′ end of the polynucleotide. In another preferred embodiment, the silane is an aminosilane, where the amino group is reactive with a multifunctional reagent or a biopolymer.

In still another preferred embodiment, the silane is an organosilane comprising a reactive group reactive with a multifunctional reagent or biopolymer, wherein the organosilane is an alkyl silane and the alkyl moiety is selected from the group consisting of an ethyl-, a propyl-, a butyl-, a pentyl-, a hexyl-, a heptyl-, an octyl-, a nonyl-, and a decylalkyl moiety, and the reactive functionality of the organosilane is covalently linked to the alkyl moiety. The alkyl moiety comprises a cyclic portion. The organosilane may also comprise an aryl moiety linking the reactive functionalities to the silane. Where the reactive groups on the silane and the target biopolymer are primary amines, the reactive groups on the multifunctional linker reagent are preferably thiocyanate groups reactive with primary amines.

Accordingly, an embodiment of the invention involves an activated microarray slide comprising a silanized surface prepared by silanizing the surface with an aminosilane in toluene in the absence of acetone or an alcohol, and a multifunctional linker reagent attached to the silane; wherein at least one pendent reactive group of the multifunctional linker reagent is a thiocyanate moiety capable of reacting with an unmodified polynucleotide or a polynucleotide modified by the incorporation of a primary amine at its 5′ end.

In yet another aspect, the invention involves a method for preparing a solid support matrix to which nucleic acids are attached in making a nucleic acid array. According to the invention, toluene is used as a solvent in silane-based modification by PDITC chemistry. The invention derives from the discovery disclosed herein that DNA which is unmodified still attaches to an activated glass solid support, such as a glass slide. The advantage of the present invention is that the use of toluene as solvent in silanization of the glass, rather than acetone as the solvent, reduces the fluorescent background and improves the signal-to-noise ratio. In addition, the modified surface of the glass slide obtained by the method of the invention promotes the preparation of microarrays having improved nucleic acid spot morphology, such as reduced overlap with adjacent spots on a densely packed microarray slide, and uniform distribution of the nucleic acid on the surface comprising the spotted region.

In another aspect, the invention involves a method of attaching a target molecule to a surface of a substrate, the method comprising providing an activated microarray slide, wherein the activated slide comprises a silanized surface prepared by silanizing with an organosilane in toluene in the absence of acetone or an alcohol, and contacting a modified or unmodified biopolymer with the surface of the activated slide under conditions causing the biopolymer to covalently or non-covalently attach to the surface of the slide.

In an embodiment, the invention involves a reacting a multifunctional linker reagent with a reactive group on the organosilane such that the multifunctional linker reagent is attached (covalently or non-covalently) to the silane leaving at least one reactive group on the multifunctional linker reagent available to react with a modified or unmodified biopolymer. Preferably, the attachment of the multifunctional linker reagent to the silane is covalent. Preferably, the reactive groups on the multifunctional linker reagent are pendant in that reaction between the linker and a modified or unmodified biopolymer is not sterically hindered.

In an embodiment, the invention involves a method of attaching a target molecule to a surface of a substrate, wherein the method comprises first providing a solid support surface comprising at least one substantially flat surface. Next, the solid support surface is silanized with a silanizing agent in toluene in the absence of acetone or an alcohol, wherein the silanizing agent comprises a reactive functionality reactive with a target biopolymer. The target biopolymer is then contacted with the surface under conditions causing the target biopolymer to become attached to the silanizing agent on the surface, thereby immobilizing the target biopolymer on the surface. Where the biopolymer is unmodified, the reactive group on the silanizing agent is reactive with a naturally occurring functionality on the biopolymer. Where the target biopolymer is modified, it is preferably modified with a reactive group that is capable of reacting with and forming an attachment to a functionality on the silanized surface of the support.

In a related embodiment, the invention involves a method of attaching a target biopolymer to a support surface of a substrate, wherein the method is like that just described except that after silanizing the surface, a multifunctional linker reagent is attached to the silane followed by attachment of the target biopolymer to the multifunctional linker. Preferably, the multifunctional linker reagent comprises a first reactive group that reacts with a functionality on the silane and a second reactive group that reacts with a functionality on the target biopolymer. The reactive groups of the silane, the multifunctional linker reagent and, optionally, a modified biopolymer are chosen to allow rapid and efficient reaction and attachment of the molecules to the surface. Preferably, the silane is an aminosilane, the linker is a diisothiocyanate compound, and the biopolymer, if modified, is modified with a 5′ primary amine. In a preferred embodiment, the silane is an organosilane, such as 3-aminopropyltriethoxysilane. In another preferred embodiment, the multifunctional linker reagent is phenylene diisothiocyanate. Optionally, the target biopolymer is unmodified prior to reaction with the silane or the linker reagent.

In another aspect, the invention involves an improved method of nucleic acid (DNA and RNA) purification from tissue samples. The method comprises, in part, a modified cesium chloride purification useful for nucleic acid preparations from tissues or cell culture, for example. The highly purified RNA according to the invention, for example, is useful for the making of probes directly from RNA without a polyA+ purification step, which step causes substantial loss of starting RNA material. The method is also useful to re-purify commercially available RNAs to improved detection sensitivity.

In one aspect, the invention involves improved methods for generating fluorescently labeled sDNA probes from small quantities of nucleic acids, particularly ribonucleic acids. In mammalian tissue, for example, approximately 1% of the total RNA is messenger RNA/polyA+ RNA. Because mRNA/polyA+ RNA is the material providing the initial template for DNA probe synthesis, it is available in very small amounts against a complex background of non-messenger RNAs (ribosmal RNA, transfer RNA, and the like). Consequently, the method of the invention for DNA probe synthesis provides an advantage because the quantities of RNA useful as a template according to the present method are 100-1000 fold less than the amounts useful in previously known methods.

According to this aspect, the invention involves a method of preparing a nucleic acid probe capable of forming a detectable complex with a target molecule, the method comprises isolating an amount of RNA from a biological sample; synthesizing a mixture of detectably labeled cDNA probes complementary to the isolated RNA in the presence of a detectably labeled deoxyribonucleotide; degrading ribonucleic acid with RNase; decreasing the average length of the labeled cDNA probes in the preparation to he from approximately 0.5 kb to approximately 2 kb by limited DNase digestion; and isolating the labeled cDNA probes. According to the invention, the isolated RNA is total cellular which includes messenger RNA. Preferably, the biological sample is selected from the group consisting of a cell, a tissue sample, a body fluid sample, and a mixture of synthetic oligonucleotides.

In an embodiment, the invention involves a method for generating fluorescently labeled sDNA probes using small quantities of total cellular RNA, where the quantities are nanograms or picograms. Such small amounts of total RNA are equivalent to low picogram or femtogram quantities of cellular messenger RNA, where mRNA is the actual template for reverse transcription to sDNA. Additional embodiments of the invention include generating fluorescently labeled DNA probes from RNA isolated from cells, such as cells in tissue or in cell culture. Where the cells are from tissue, such as diseased human tissues, tumor cells are microdissected nearby non-tumor cells in the diseased tissues. Tissue from which total RNA is isolated includes non-diseased and diseased tissue and further includes fresh tissue, frozen tissue, and formalin-fixed paraffin-embedded tissue. According to the invention, the amount of isolated total cellular RNA is from approximately 0.01 pg to and including approximately 10 mg, 1 pg to and including 10 μg, 100 pg to and including 100 ng, and 500 pg to and including 10 ng.

In an embodiment of the method of preparing a cDNA probe, the invention involves the additional steps of synthesizing double stranded DNA from messenger RNA in the isolated total cellular RNA, followed by synthesizing RNA complementary to the double stranded DNA. It is understood that cellular DNA may be isolated from the biological sample and used as starting material for a DNA or cRNA probe according ot the invention.

In another embodiment, the method of preparing a cDNA probe involves labeling the synthesized cDNA probe by incorporating a detectably labeled deoxyribonucleotide. Preferably, the labeled deoxyribonucleotide is dUTP. In a related embodiment the synthesizing of the labeled cDNA probe is performed in the presence of labeled and unlabeled dUTP and in the absence of dTTP.

Preferably, the detectable label is a fluorescent molecule and the detection is by fluorescence emission. Other methods of detection may be used, including, but not limited to, radioisotope labeling and detection, as well as mass spectrometry (see, for example, Marshall, A. and Hodgson. J. Nature Biotechnology 16:27-31 (1998)).

Preferably, where the biological sample is a cell culture or tissue sample, the cells of interest from the culture or tissue are specifically extracted from the biological sample generally independent from surrounding cells that of a different type or different disease state that are present nearby in the tissue or culture. Preferably, a control sample (e.g. a sample of normal tissue) comprises cells removed from the tissue source by laser capture microdissection, wherein the cell source is selected from the group consisting of untreated tissue, frozen tissue, paraffin-embedded tissue, stained tissue, and cell culture. Preferably, a test sample (e.g. a sample of diseased tissue) comprises cells removed from the tissue source by laser capture microdissection, wherein the cell source is selected from the group consisting of untreated tissue, frozen tissue, paraffin-embedded tissue, stained tissue, and cell culture.

In another aspect, the invention involves a method for generating fluorescently labeled cRNA probes from small quantities of total cellular RNA, where the quantity is nanograms or picograms. Such small amounts of total RNA are equivalent to low picogram or femtogram quantities or cellular messenger RNA, where mRNA is the actual template for generation of double stranded DNA followed by transcription to cRNA. Additional embodiments of the invention include generating fluorescently labeled cRNA probes ultimately from RNA isolated from cells, such as cells in tissue or in cell culture. Where the cells are from tissue, such as diseased human tissues, tumor cells are microdissected nearby non-tumor cells in the diseased tissues. Tissue from which total RNA is isolated includes non-diseased and diseased tissue and further includes fresh tissue, frozen tissue, and formalin-fixed paraffin-embedded tissue.

In an embodiment, the invention involves a method of preparing a nucleic acid probe capable of forming a detectable complex with a target molecule, where the method comprises isolating an amount of RNA from a biological sample; synthesizing a mixture of detectably labeled complementary RNA probes by synthesizing double stranded DNA from messenger RNA in the isolated RNA, followed by synthesizing RNA complementary to the double stranded DNA in the presence of a detectably labeled ribonucleotide; and isolating the labeled cRNA probes. Optionally, sDNA is prepared by synthesizing cRNA complementary to the double stranded DNA, but in the absence of fluorescent deoxynucleotides, followed by synthesizing sDNA probes from the cRNA in the presence of labeled fluorescently labeled deoxynucleotides and using random primers. Random priming controls the length of the sDNA probes. Preferably, the average length of the labeled sDNA probes is from approximately 0.5 kb to approximately 3 kb, preferably from approximately 0.5 kb to approximately 2 kb. For cDNA probes, the average length is altered, if necessary, by mild Dnase digestion. For cRNA probes the average length of the labeled probes is decreased by mild RNase digestion or limited fragmentation by resuspending the precipitated, labeled cRNA probes in 40 mM tris-acetate, pH 8.1, 100 mM potassium acetate, 30 mM magnesium acetate, followed by heating at 70° C. for 10 min. Preferably, the isolated RNA is total cellular RNA. Preferably, the biological sample is selected from the group consisting of a cell, a tissue sample, a body fluid sample, and a mixture of synthetic oligonucleotides.

In another embodiment, the method of preparing a cRNA probe involves labeling the synthesized cRNA probe by incorporating a detectably labeled ribonucleotide. Preferably, the ribonucleotide is UTP. Preferably the detectable label is a fluorescent molecule. In a related embodiment the synthesizing of the labeled cRNA probe is performed in the presence of labeled and unlabeled UTP.

In another aspect, the invention involves a method for generating fluorescently labeled sDNA (sense strand DNA) probes from small quantities of total cellular RNA, where the quantity is nanograms or picograms. Such small amounts of total RNA are equivalent to low picogram or femtogram quantities of cellular messenger RNA, where mRNA is the actual template for generation of double stranded DNA followed by transcription to cRNA as an amplification step and without incorporation of label in the cRNA. To generate labeled sDNA probes, the cRNA is reverse transcribed in the presence of fluorescent nucleotides, preferably fluorescent dUTP nucleotides.

In still another aspect, the invention involves a method for generating fluorescently labeled sDNA probes from total cellular RNA without amplification. According to the invention, total cellular RNA was used as the starting material for first strand DNA synthesis. Labeled sDNA probes are prepared by direct synthesis of a second strand DNA from the first strand using the Klenow fragment of DNA polymerase I.

According to the invention, the amount of isolated RNA useful for probe synthesis (cDNA, cRNA, or sDNA probes) is from approximately 0.01 pg to and including approximately 10 mg. 0.5 pg to and including 1 ng, 1 pg to and including 500 μg, 10 pg to and including 10 μg, 100 pg to and including 100 ng, and 500 pg to and including 10 μg.

According to the methods of preparing nucleic acid probes, the invention involves deriving control nucleic acid probes from total cellular RNA from a control sample comprising a single or pooled mixture of samples of similar tissue type, tissue origin, developmental stage, or the like. For example, the control sample comprises samples of normal tissue of the same organ from different donors or derived from the same tissue type from the same or different donors. For example, in one embodiment, the invention involves pooling multiple epithelial tissues as a control sample from which a control nucleic acid probe is derived for use in detecting gene expression or copy numbers in comparison with expression or copy numbers in a test carcinoma. In a related embodiment, the control sample is a mixture of cells from one or more cell cultures, where the cells are pooled prior to isolation of total cellular RNA. A control nucleic acid probe generated from pooled cell cultures is compared to a test nucleic acid probe in its ability to complex with a target molecule. According to the invention, the test nucleic acid probe may also be derived from a mixture of test tissue cell samples or test cell culture samples.

In another aspect, the invention involves a method of preparing glass slides for application of nucleic acid in a microarray pattern, wherein the method involves cleaning the slides with detergent and alkali; silanizing the slides with an organosilane in toluene in the absence of acetone or an alcohol; optionally reacting the organosilane with a multifunctional linker reagent capable of reacting with a functional group of the organosilane and a target molecule; followed by contacting the activated surface (comprising the reactive organosilane attached to the surface or, if present, the multifunctional linker reagent attached to the organsilane) under conditions that cause the target molecule to be attached to the surface by covalent or non-covalent attachment. The method also involves the steps of washing the silanized slides in solvents including toluene, methanol, water, and methanol to remove unreacted compounds and drying the slides after the attachment of the organosilane, the multifunctional linker reagent, and the target molecule.

In an embodiment, the toluene is at least 50% of the solvent in the silanizing step, preferably at least 80%, more preferably at least 90%, more preferably at least 95%, still more preferably at least 99%, and most preferably the toluene is at least 99% of the solvent in the silanization reaction mixture and is dried by standard techniques and of standard purity suitable for efficient silanization reactions and minimal background fluorescence during subsequent detection steps according to the invention.

In another embodiment, the invention involves a method of attaching a modified target polynucleotide to a microarray solid support, wherein the method comprises obtaining a nucleic acid primer comprising a reactive group covalently attached to its 5′ end by a linker, wherein the primer is complementary to sequences outside the target polynucleotide; amplifying the target polynucleotide by polymerase chain reaction to produce modified target polynucleotide comprising the reactive group; obtaining an activated microarray comprising on a surface a surface reactive group capable of reacting with the modified target polynucleotide reactive group, wherein the microarray solid support is pretreated by silanizing the surface with an organosilane in toluene; contacting the modified target polynucleotide with the microarray solid support, whereby the modified target polynucleotide reactive group and surface reactive group react covalently attaching the modified target polynucleotide to the microarray solid support. Preferably, the modified target polynucleotide reactive group comprises a primary amine and the surface reactive group comprises a isothiocyanate moiety.

In another aspect, the invention invloves a method of analyzing a biopolymer target on a microarray, wherein the method comprises providing a microarray slide comprising a target biopolymer attached to a silanized substrate surface, prepared by silanizing with an organosilane in toluene in the absence of acetone or an alcohol; contacting the attached target molecule with an agent capable or forming a detectable complex with the target molecule under conditions that allow formation of a detectable complex; detecting formation of a detectable complex; determining the amount of a detectable complex formed.

In an embodiment, the agent capable of forming a detectable complex comprises (1) a control mixture of nucleic acid probes comprising a first detectable label, wherein the probes are prepared from nucleic acid isolated from a control sample, and (2) a test mixture of nucleic acid probes comprising a second detectable label, wherein the probes are prepared from nucleic acid isolated from a test sample, wherein the first and second detectable labels, and the nucleic acid molecules to which they are attached, can be detectably distinguished one from the other for ease of determining the presence of, and optionally, the relative amounts of the probes in a mixture or the amounts of control and test probes forming complexes with a particular target molecule on a microarray. The method further involves pooling the control probes and the test probes; contacting the pooled probes with a target molecule on a microarray slide prepared according to the invention under conditions that allow the formation of specific detectable complexes between a control probe or a test probe; and comparing the amount of detectable complex formed between the target molecule and the control probes relative to the amount of complex formed between the target molecule and the test probes. Individual probes can also be singly hybridized to a microarray to generate quantitative expression data that can be compared to data from other singly hybridized or pooled probe hybridized microarrays. Preferably the target molecule is a target polynucleotide and probes are either cDNA probes cRNA probes, or sDNA probes, or a combination of these. Preferably the label is optically detectable, such as by fluorescence emission. Preferably the complex formation between the target molecule and the probes occurs in the absence of detergent, although a subsequent washing step optionally involves a solution comprising a detergent. In an embodiment of the invention, sodium dodecyl sulfate (SDS) is eliminated from the hybridization solution in which a complex is formed between the target molecule and the probes. In yet another embodiment, hybridization is performed in the presence of an alkylammonium salt, DMSO and formamide to further improve complex formation.

In another aspect, the invention involves a method of hybridizing a detectable polynucleotide probe to a target polynucleotide on a support surface, the method comprising: (a) contacting the probe with denatured target polynucleotide on the support surface in the absence of detergent; and (b) detecting formation of a complex between the target polynucleotide and the delectably labeled polynucleotide probe. In an embodiment of the invention, sodium dodecyl sulfate (SDS) is eliminated from the hybridization step. In another embodiment of the invention, hybridization efficiency is improved by using a hybridization solution comprising formamide and one or more or an alkylammonium chloride (preferably tetrameythlammonium chloride, or tetraethylammonium chloride, or both) and dimethylsulfoxide (DMSO).

According to the invention, the test sample and control sample differ from each other according to one or more of developmental state, disease state, pre-disease state, cell type, sample source, and experimental treatment conditions. Optionally, according to the invention, the control sample comprises a mixture of samples that differ from the test sample according to one or more of developmental state, disease state, cell type, sample source, and experimental treatment conditions. Optionally, according to the invention, the test sample comprises a mixture of samples that differ from the control sample according to one or more of developmental state, disease state, cell type, sample source, and experimental treatment conditions.

In an embodiment of the invention, the target molecule is a polynucleotide and the nucleic acid isolated from the test sample and the control sample is RNA, and wherein the comparing provides a measure of target polynucleotide expression in the test sample relative to target polynucleotide expression in the control sample. Preferably, the relative measure of target polynucleotide expression indicates a disease state in the test tissue sample, and the disease state is selected from the group consisting of all forms of cancer, cardiovasular disease, neurological disease, inflammation, and any disease that may be characterized by an alteration in gene expression relative to a non-disease state. In a related embodiment, the relative measure of target polynucleotide expression indicates a predisease state in the test tissue sample. In another related embodiment, the target molecule is a polynucleotide and the nucleic acid isolated from the test sample and the control sample is DNA, and wherein the comparing provides a measure of number of copies of the target polynucleotide in cells of the test sample relative to target polynucleotide copies in the control sample, and the relative measure of the number of copies of target polynucleotide indicates a disease state or a pre-disease state in the test tissue sample.

### DESCRIPTION OF THE EMBODIMENTS

Definitions

As used herein, the terms “attached,” “attachment,” “bound,” and like terms refer to a physical or chemical linkage between at least two molecules. For example, where the attachment is between a target molecule and a substrate surface, the attachment is preferably a covalent chemical bond. Where the attachment is between a target molecule to be immobilized on a substrate surface and a reactive linker reagent on the surface, the attachment is preferably covalent. Electrostatic, hydrophobic, hydrophilic, or other noncovalent chemical bonds may form the attachment, however, if such noncovalent bonds prevent migration of the target molecule from its initial point of contact on the support surface. Where the binding is within a complex between a target molecule and an agent (a probe) capable of complexing with the target molecule, the binding is preferably eletrostatic, hydrophobic, hydrophilic, or other noncovalent binding.

As used herein, the term “biopolymer” refers to a target molecule of interest that may be attached to a substrate according to a procedure appropriate to the structure of the biopolymer. Optionally, the bioplymer is a nucleic acid sequence, including a single stranded or double stranded polynucleotide, where the polynucleotide may be RNA, DNA, or PNA (peptide nucleic acid, wherein the nucleotide backbone is a peptide backbone). Where the biopolymer is a protein, such as a ligand, a receptor, an antibody, cell surface protein, and the like, the probe is, for example, a receptor, ligand, antibody polynucleotide, or other biopolymer or smaller molecule capable of forming a complex with the target protein. Preferably, the biopolymer is known, knowable, determinable, or otherwise identifiable.

As used herein, the term “detergent” refers to a surfactant useful for causing or enhancing denaturation of target molecules as well as enhancing wetting of the support surface during hybridization. Non-limiting examples of detergents includes sodium dodecylsulfate (SDS), Triton X-100, Nonidet P-40, and Tween-20.

As used herein, the term “discernable,” or “distinguishable,” with regard to detection of a complex formed by a target molecule with a control probe versus a complex formed by a target molecule and a test probe, refers to the ability to detect a control complex as different from a test complex by direct visual detection or assisted detection through the use of a detecting instrument. For example, a complex comprising a control probe labeled with a first fluorescent dye is discernable from a complex comprising a test probe labeled with a second fluorescent dye where the first and second dyes emit at different wavelengths.

As used herein, the phrase “disease state” refers to an abnormal state of a cell or a tissue, where the abnormal state in a living animal or plant results in illness or death. Non-limiting examples of a cell or tissue in a diseased state include all forms of cancer, cardiovasular disease, neurological disease, inflammation, and any disease that may be characterized by an alteration in gene expression relative to a non-disease state.

As used herein, the term “dye 488” refers to a dUTP- or UTP-derivatized fluorochrome, where the fluorescent chromophore excites at a wavelength of 488 nm and emits around a peak wavelength of 530 nm. The Alexa Fluor 488 Dye (Molecular Probes, Inc.) is an example of such a dye. Commonly used fluorescein dye also emits at this wavelength, the green region of the visible spectrum, and is useful in the invention. The preferred dye for use in the present invention is the most intensely emitting chromophore available to the user, which is more photostable than fluorescein, and which is relatively unaffected by variations in the pH range used in microarray hybridization analysis (for example between pH 4 to 10). In addition. Alexa Fluor Dye 488 is advantageous because it has a narrower emission spectrum which results in reduced fluorescence interaction with dye 546, thereby allowing improved signal-to-noise ratios.

As used herein, the term “dye 546” refers to a dUTP- or UTP-derivatized fluorochrome, where the fluorescent chromophore excites at a wavelength of 546 nm and emits around a peak wavelength of 590 nm. The Alexa Fluor 546 Dye (Molecular Probes, Inc.) is an example of such a dye. Commonly used Cy3 dye and tetramethylrhodamine (TRITC and TAMRA) also emit at this wavelength, the red region of the visible spectrum, and are useful in the invention. The preferred dye for use in the present invention is the most intensely emitting chromophore available to the user.

As used herein, a “glass slide,” with respect to microarray solid support, refers to a piece of planar silica-based glass of a size, shape, and thickness to allow convenient manipulation of the slide during microarray preparation and subsequent microarray analyses.

As used herein, a “multifunctional linker reagent” refers to a molecule capable of binding to another molecule, polymer, or surface while also capable of binding to still another molecule, polymer, or surface. For example, a linker molecule comprises at least two reactive groups capable of such binding to two or more other molecules. According to the invention, examples of linker molecules include an organosilane capable or binding to a surface (such as a glass surface) through an alkoxy silyl moiety, and capable of reacting with a target molecule or another linker molecule. Another linker molecule may be a bifunctional reagent capable of reacting with a reactive functionality on a surface-bound organosilane as well as being capable of reacting with an unmodified or modified target molecule.

As used herein, the term “normal tissue” refers to tissue in which no discernable disease is observed according to standard medical diagnostic methods, or at least a disease state of a test sample is not present in the control normal tissue sample.

As used herein, the term “nucleic acid” refers to a deoxyribonucleoside or ribonucleoside, or a deoxyribonucleotide or ribonucleotide polymer in either single-stranded or double-stranded form. The term further encompasses nonnatural analogs of natural nucleotides, such as peptide nucleic acids.

As used herein, the term “oligonucleotide” refers to a single-stranded nucleic acid sequence comprising from 2-1000 nucleotides in length, 10-750 nucleotides, 20-500 nucleotides, 50-400 nucleotides, or 50-200 nucleotides in length. An oligonucleotide may be chemically synthesized by standard techniques in the art of nucleic acid synthesis. Such techniques included, but are not limited to solid phase synthesis followed by release of the oligonucleotide from the solid phase prior to attachment to a microarray slide, and solid phase synthesis on a microarray slide (see, for example, U.S. Pat. No. 5,445,934).

As used herein, the phrase “pre-disease state” refers to an abnormal state of a cell or a tissue, where the abnormal state in a living animal or plant may not be detectable. The pre-disease state in the animal does, however, predispose the animal to eventual development of a disease state. Non-limiting examples of a pre-disease state include abnormal levels of genetic material, such as gene copy numbers, abnormal sequences of genetic material, such as disease-associated polymorphisms, changes in gene expression that frequently precede a disease state, as well as genetic profiling or tumor subtypes (see, for example, Hacia, J. G., Nature Genetics 21(Suppl):42-47 (1999); Heiskanen, M. A. et al., Cancer Research 60:41-46(2000); Pollack, J. et al., Nature Genetics 23:41-46 (1999); DeRisij, et al., Nature Genetics 14:457-460 (1996); Berns, A., Nature 403:491-492 (2000); and Alizadeh, A. A. et al., Nature 403:503-511 (2000); Marx, J., Science 289:1670-1672 (2000)).

As used herein, the term “probe” refers to an agent, preferably a detectably labeled agent, capable of forming a complex with a target molecule immobilized on a surface. Where the target molecule is a polynucleotide, the probe is another polynucleotide, a nucleic acid specific binding protein or antibody, or other nucleic acid binding molecule. For example, the probe is another polynucleotide such as RNA or DNA or a peptide nucleic acid (PNA, nucleic acid having a peptide backbone). Where the target molecule is a protein, such as a ligand, a receptor, an antibody, cell surface protein, and the like, the probe is, for example, a receptor, ligand, antibody, polynucleotide, or other biopolymer or smaller molecule capable of forming a complex with the target protein. Preferably, the complex formed between the target molecule and the agent is specific and detectably distinguishable from complex formation with other target molecules in a microarray. It is noted that the term “probe” is occasionally used to describe the immobilized biopolymer attached to a microarray surface, For the purposes of the present disclosure, the term “probe” will be used to refer to a labeled molecule capable of forming a complex with an immobilized molecule (the “target” as used herein) on a support surface.

As used herein, the phrase “reactive functionality at the 5′ end” of a polynucleotide, refers to a reactive functionality (chemically reactive moiety of a chemical compound) attached directly or indirectly via a linker, where the site of attachment is within 50 bp, 20 bp, 10 bp, 5 bp, or 2 bp of the 5′ end of the nucleic acid sequence. Preferably, the reactive functionality is within the 5′ terminal nucleotide, either on the nucleotide base or on the deoxyribose.

As used herein, the term “silanizing,” with respect to activating microarray slides, refers to reacting a silane with a substrate surface such that the silane attaches to the substrate surface. According to the invention, silanizing a microarray substrate surface refers to the reaction in which the silane reacts with a siloxy group on the surface. According to the invention, the silanizing occurs in toluene and in the absence of acetone or an alcohol. The toluene of the silanizing reaction is preferably substantially dry (such as commercially available reagent grade toluene). According to the invention, acetone or an alcohol may contact the microarray slide during other, non-silanizing reactions or washes, but contact with acetone is preferably limited to 3 hours or less, preferably 2 hours or less, followed by thorough drying to remove the acetone. Preferably, the surface comprises silica. More preferably the surface is a silica-based glass. According to the invention, the silane preferably comprises a plurality of reactive functionalities (or reactive groups), wherein at least one reactive group is capable of reacting with the surface causing the silane to be attached to surface, and at least one other reactive functionality which is capable of reacting with a reactive functionality of a target molecule, thereby attaching the target molecule to the silane and, ultimately, to the substrate surface. Optionally, the target molecule attaches to a multifunctional linker reagent that, in turn, attaches to the silane via reactive functionalities on the multifunctional linker reagent and the silane. It is understood that the linker reagent may comprise multiple linker reagent monomers.

As used herein, the term “spotting” or “tapping,” with respect to depositing a target molecule on a microarray substrate surface, refers to contacting the surface with a device, such as a microarray printing pin, containing a target molecule such that the target molecule is deposited on the surface and is in contact with the surface of the microarray. Preferably, the spotting or tapping is via a capillary or other tube (such as within the printing pin) capable of depositing a small volume of solution comprising target molecule on the surface, wherein the volume is 1 μl or less, 100 nl or less, 10 nl or less, 5 nl or less, 2 nl or less, 1 nl or less, or 0.5 nl or less. Preferably the spot formed by depositing the target molecule solution on the surface is separated from other spots on the microarray such that subsequent hybridization or other reaction on the array is not adversely affected by reactions on neighboring or nearby spots. Preferably, the spot is from 50-500 microns, from 75-300 microns, or from 100-150 microns in diameter.

As used herein, the term “substrate” refers to a solid support to which, according to the invention, a target molecule is attached, either directly or indirectly, by coupling one or more linker molecules to the substrate and ultimately to the target molecule. Non-limiting examples of substrate according to the invention include polymeric materials, glasses, ceramics, natural fibers, silicons, metals, and composites thereof. The substrate has at least one surface that is substantially flat. As used herein, the phrase “substantially flat” with regard to a substrate surface refers to a surface that is macroscopically planar for more convenient application of target molecules in a two-dimensional array. Alternatively, the substrate may have a spherical surface or an irregular surface to which a target molecule is attached and to which target molecule a probe may be complexed for detection of such complexes.

As used herein, the term “unmodified,” as used with respect to a target biopolymer such as target polynucleotide of the invention, refers to a polynucleotide that lacks reactive functionalities added or incorporated into a polynucleotide during or after its synthesis, isolation, or other preparation. Generally, according to the invention, a biopolymer's reactive functionality, the addition of which modifies a biopolymer, is one that allows attachment of the biopolymer to a microarray substrate. A unmodified biopolymer, on the other hand, lacks such a functionality added for the purpose of attaching a target biopolymer to a surface directly or indirectly through a linker molecule. Stated another way, an unmodified biopolymer is one in a native state wherein the functionalities (reactive or otherwise) that are present in the molecule are native to a naturally occurring like biopolymer. Where an unmodified target biopolymer covalently attaches to a microarray slide, the unmodified biopolymer does so at functionalities typical of a naturally occurring biopolymer or a biopolymer as it is isolated from a cell. Where the unmodified biopolymer is an unmodified polynucleotide, such as RNA, DNA or PNA, the unmodified polynucleotide attaches to the substrate at functionalities typical of a naturally occurring nucleic acid base, a polynucleotide backbone, or a polypeptide backbone.

### DESCRIPTION OF THE DRAWINGS

FIG. 1 is a photograph or microarray images generated using fluoroprobes synthesized by the method of the invention from 1-5 ng total RNA from microdissected colon tumor cells.

FIGS. 2A is a photograph of microarray images generated using fluoroprobes synthesized by the method of the invention from 5 μg total RNA isolated from formalin-fixed paraffin-embedded liver tissue. FIG. 2B is a photograph of microarray images generated using fluoroprobes synthesized by the method of the invention from 5 μg total RNA isolated from fresh frozen adult liver. Probes generated from paraffin-embedded starting material were comparable in detection sensitivity to probes generated from fresh frozen tissue (compare FIG. 2A and FIG. 2B). FIG. 2C is a photographic image of a microarray analysis from a formalin-fixed paraffin-embedded colon tumor, 4 μg total cellular RNA starting material. FIG. 2D is a scatter plot of the fluorescence intensities from microarray analysis of colon tumor RNA isolated from the same patient, a fresh-frozen sample (X axis) versus a formalin-fixed paraffin-embedded sample (Y axis).

FIG. 3A is a photograph of microarrays showing hybridization of probes synthesized from breast tumor RNA. FIG. 3B shows hybridization of probes synthesized from epithelial tissue RNA pool reference sample. In general, gene expression is quantified by comparison of the intensity and wavelength emitted from each spot for test versus control samples.

FIGS. 4A, 4B, and 4C are photographs of microarrays showing successful detection of hybridized sDNA probes synthesized from various amounts of total cellular RNA starting material from an ovarian carcinoma cell line. The figures display the results of a 1-color analysis of fluorescence intensity achieved on a microarray according to the invention when the amount of total cellular RNA starting material was limited to 200 pg (FIG. 4A), 20 pg (FIG. 4B), and 2 pg (FIG. 4C).

### EXAMPLES

The following examples are offered by way of illustration and not by way of limitation. The examples are provided so as to provide those of ordinary skill in the art with a complete disclosure and description of how to make and use the compounds, compositions, and methods of the invention and are not intended to limit the scope of what the inventors regard as their invention. Efforts have been made to insure accuracy with respect to numbers used (e.g. amounts, temperature, etc.), but some experimental errors and deviation should be accounted for. Unless indicated otherwise, temperature is in degrees C. (° C.). The disclosures of all citations in the specification are expressly incorporated herein by reference.

### Example 1

### Nucleic Acid Preparation for Microarray Analysis

The invention is useful for detecting the presence of nucleic acids in any mixture of nucleic acids. The present invention finds its preferred use, however, in the detection and quantification of gene expression in tissue samples, a medium in which detection of gene expression has heretofore posed distinct challenges. The present invention solves this problem by providing a method of improving the detection limit for gene expression in tissue samples.

Collecting Cells for Control or Test Samples: Microarray analysis allows the direct comparison of cellular states between test and control samples of cells, tissue, body fluids, and the like. Such comparisons are optimized when the test or control sample comprises exclusively or substantially only the cells of interest. For example, a diseased tissue, such as cancer tissue, frequently comprises cancerous cells that have infiltrated an area or normal cells. Thus, a sample of cancerous tissue will often contain a mixture of normal and diseased cells and may also include several cell types found in the tissue or associated with the cancer, such as cells associated with the inflammatory and immune responses to cancer. Preferably, a sample comprises only those cells important to the analysis. According to the present invention, it is preferred that the test sample comprises a collection of cells collected specifically by cell type or other desired state such that contamination of the sample by cells of a different type or state are excluded.

The technique of laser-capture microdissection (LCM) is preferred for cell collection (see, for example, Emmert-Buck, M. R. et al., Science 274:998-1001 (1996); Simone, N. L. et al., Trends in Genetics 14:272-276 (1998); Glasow, A. et al., Endocrine Research 24:857-862 (1998); WO 002892 (priority date Nov. 5, 1998); Luo, L. et al., Nature Medicine 5:117-122 (1999); and Arcturus Engineering, Inc., www.arctur.com, last visited Mar. 20, 2001). LCM was developed to provide a method for obtaining pure populations of cells from specific microscopic regions of tissue sections under direct visualization (Simone, N. L. et al. supra). For the purposes of the invention and the present examples, the cells of interest were transferred to a polymer film activated by laser pulses, a technique that maintained the integrity of the RNA. DNA, and proteins of the collected cells. The transferred cells were used for the isolation of total cellular RNA for subsequent use in the preparation of control nucleic acid probes and test nucleic acid probes. The LCM device used for the examples disclosed here was from Arcturus Engineering, Inc., (Mountain View, Calif., USA).

Isolation and Purification of Nucleic Acids from Biological Samples: The nucleic acid preparation method of the invention involves a cesium chloride density gradient protocol. It is useful for collecting both RNA and DNA from tissue samples of limited size and from a variety of tissue sources including, but not limited to tumor tissue of epithelial origin. RNA obtained by this method is sufficiently pure to allow the direct synthesis of probes from the RNA and allows improved probe labeling. This method was found to particularly useful for isolating RNA from tissues such as liver or fetal heart which are rich in contaminating carbohydrates. Additionally, the method of the invention is useful for purifying commercially obtained RNAs, thereby allowing for improved probe synthesis and labeling of RNAs from commercial sources.

Purification of nucleic acids from tissue samples is provided as an example of the method of the invention and its usefulness. Tissue samples from normal tissue (or a pool of normal tissues) is designated “control tissue” or “control sample” herein. Tissue samples from a diseased tissue, such as tumor tissue, is designated “test tissue” or “test sample” herein. Unless otherwise indicated, the preparation of control and test samples is the same in the present example.

Tissue, either test or control tissue, was ground to powder in liquid nitrogen, followed by douncing 8-10 times in at least 10 volumes of lysis buffer (4M guanidine thiocyanate, 25 mM sodium citrate, 0.5% N-lauryl sarcosine) to provide a tissue lysate. For example, to approximately 100 mg of tissue, approximately 1-2 ml of lysis buffer was added. The lysate was centrifuged at 12,000 rpm in an SS34 rotor (approximately 12,100×g; Beckman Instruments, USA) for 10 min. to remove insoluble material. The clarified lysate was then layered on top of 5.7M cesium chloride/50 mM EDTA pH 8 (designated “CsCl” for convenience) at a volume-to-volume ratio of 1:2.25 CsCl:lysate.

The CsCl:lysate preparation was centrifuged at 150,000×g for at least 12 hours to sediment RNA from the suspension. For tubes compatible with a SW 55 rotor (Beckman Instruments, USA), 3.5 ml lysate was layered on 1.5 ml CsCl for a total volume of 5 ml. When a TLS 55 rotor (Beckman Instruments) was used for smaller samples of 50-200 mg tissue, 1.4 ml lysate was layered onto 600 μl CsCl and centrifuged.

The lysate was removed and retained for DNA purification. The RNA pellet was observed as a glassy precipitate at the bottom of the centrifuge tube. After removing cesium chloride solution from the centrifuge tube and washing the pellet with highly purified water, the RNA pellet was resuspended in a volume of TE (10 mM Tris, 1 mM EDTA, pH 8-8.5) sufficient, to resuspend the pellet. Resuspension may be slow, requiring 12 or more hours to resuspend large pellets in small volumes.

Resuspended RNA was extracted by standard phenol:chloroform extraction techniques. The RNA was precipitated by the addition of 0.1 volume (relative to the aqueous layer) of 3M sodium acetate and 3 volumes of ethanol. The precipitate was washed with 70% ethanol, followed by washing with 95% ethanol. The pellet was dried and resuspended in highly purified water, such as double-distilled and deionized water or the like.

Where the sample was cells in culture, the method of purifying nucleic acids was modified as follows. To cells harvested from a 10 cm culture plate or a 15 cm plate, 2 ml or 3.5 ml, respectively, of the lysis buffer was added. Lysate was collected using a syringe equipped with an 18 gauge needle. Low-speed centrifugation at 12,000 rpm in an SS34 rotor may be omitted for the preparation of cultured cell lysate. Following collection of lysate, the procedure for nucleic acid purification from cultured cells was the same as that for tissue samples.

The retained DNA-containing lysate was doubled in volume with highly purified water. Material was extracted by standard phenol:chloroform extraction techniques leaving DNA in the aqueous later. DNA was precipitated by the addition of 0.7 volume isopropanol. The precipitate was pelleted at 13,000 rpm in a SS34 rotor (Beckman Instruments), for example, and mixed with a minimum amount of TE to resuspend the pellet.

The purified and resuspended RNA and DNA are useful for the preparation of probes for microarray analysis. The ability to isolate both RNA and DNA in a highly purified from a tissue sample is particularly useful in permitting correlation and comparisons between the number of gene copies (as DNA) and the level of expression (as RNA), for example.

### Example 2

### Preparation of Microarray Probes

The protocol disclosed herein for the preparation of a microarray probe is useful to analyzing very small quantities of RNA as starting material for probe synthesis. The protocol is particularly useful to generate mixtures of cDNA probes or sDNA probes from tumor cells isolated from heterogeneous tumor tissue by laser capture microdissection, for example. The number of tumor cells thus isolated is usually quite small, yet as few as 100 cells, even 10 cells, and as few as one cell is a sufficient source of RNA for gene expression analysis due to the surprising sensitivity available using the compositions and methods of the invention. The present method is also useful for probe synthesis using RNA isolated from non-microdissected cells, but is generally, although not exclusively, most useful when the quantities of RNA are limiting. The probes generated by the method of the invention are reliably sensitive even when the amount of RNA starting material is very small. For example, the invention relates to probe synthesis from as little as 2 pg-10 ng isolated total cellular RNA, which represents approximately 20 fg-100 pg messenger RNA, an amount that is approximately 1000-fold less than currently available techniques can analyze.

According to the present invention, two variations for probe synthesis are disclosed, where the variations depend on the amount of isolated total cellular RNA available. For quantities of total RNA from 500 ng-5 μg, inclusive, a direct labeling protocol is used. For quantities of RNA as small as 500 pg-10 ng of total RNA, probes are generated by a single round of amplification by in vitro transcription. For extremely small amounts or total cellular RNA (e.g. 0.01-10 pg total cellular RNA, preferably about 1-10 pg, more preferably about 1-2 pg, equivalent to the total RNA from a single cell), the initial amplification by in vitro transcription may be performed as described, or performed for a longer incubation period (e.g. for 12 hours), or performed twice to generate sufficient material for sDNA probe or cRNA probe synthesis. For each embodiment or the invention, cDNA probe, sDNA probe, or cRNA probe synthesis involves the incorporation of fluorochromes.

Before cDNA probe, sDNA probe, or cRNA probe synthesis, the RNA may be purified by micro-CsCl centrifugation or by direct precipitation of unquantified nucleic acid. For example, these purification protocols were particularly useful when working with microdissected tissue samples.

This example discloses the use of commercially available modified fluorescent dyes (the Alexa series of fluorescent dyes, Molecular Probes, Inc., Eugene, Oreg., USA) in a 2-color or one-color microarray analysis based on cDNA probes prepared directly by reverse transcription of isolated RNA purified by the method disclosed in Example 1. Similarly, cRNA probes and sDNA probes were prepared with an intermediate step of double stranded DNA synthesis from isolated RNA, followed by transcription, then, where a sDNA probe is desired, by synthesis of a labeled DNA probe using reverse transcriptase, labeled deoxyribonucleotides, and random primers. The method of probe preparation disclosed in this example is robust and highly sensitive, allowing the user to begin with as little as 500 pg-10 ng total RNA.

Preparation of Labeled DNA Probes:

The following procedures disclose non-limiting examples of methods of preparing a detectably labeled DNA probe for use in the present invention. In each example of probe synthesis, the starting material was total cellular RNA isolated from a tissue sample. As these examples demonstrate cDNA probes were prepared from RNA with no intermediate amplification or only 1 or 2 rounds of amplification. sDNA probes were prepared by reverse transcription from unlabeled cRNA. sDNA probes were also prepared from larger amounts of starting total cellular RNA by direct second strand synthesis with label incorporation. cRNA probes were prepared from cDNA.

Problems to be Solved in Developing an Improved Method of Preparing Labeled Nucleic Acid Probes:

Detection sensitivity relies, in part, on the ability to generate a maximally labeled (“hot”) probe without exceeding the solubility limits For the DNA/chromophore complex. The solubility or the DNA/chromophore complex is affected by probe labeling density and probe length. Labeling density is defined as the number of chromophores per specified DNA fragment length. Labeling density was found as part of the invention to be correlated with total labeling efficiency, and therefore, correlated with the ratio of labeled probe to unlabeled probe. This ratio is readily estimated by probe intensity visualized on a nucleic acid sequencing gel. This technique was useful for evaluating the probes for approximate labeling density, molecular weight or fragment length. In a related observation, it was found that probe solubility was inversely correlated with labeling efficiency, i.e. as the number of fluorochromes was incorporated into a probe, its solubility decreased. Thus, the visualization of labeling efficiency on a sequencing gel also provided an indirect estimation and prediction of probe solubility.

The length and, hence the molecular weight, of the probes was controlled by mild DNase digestion. Preferably the DNase digestion is performed for a time and under conditions that yield an average probe length of less than 5 kb, more preferably in the range from 0.5 kb-2 kb, inclusive, after digestion. Gel electrophoresis may be used to evaluate the degree of probe digestion. Redigestion by DNase can he performed if the average probe length is longer than the target average length.

Probes were evaluated for labeling density on an ABI 373A DNA sequencer (Applied Biosystems, Inc., USA) or other phosphoimaging or fluorescent imaging device. The use of fluorescein- and rhodamine-related dyes was useful because the different emission wavelength of each dye allowed separate detection of the labeling density for each dye. Labeling density was estimated by correlation with ratio of labeled to unlabeled probe, such that the fluorescence intensity of the probe mixture on a sequencing gel provides an indication of the labeling density. Other dyes are, of course, useful in the method of the invention. Preferably, the dyes have emission maxima that do not directly overlap and allow the separate and quantitative detection of chromophores in a probe/microarray complex.

The solubility of a labeled probe was determined directly using a charge coupled imaging device (a “CCD imager”). Solubility was also predicted by correlation with labeling density (e.g. the ratio of labeled to unlabeled probe) because an increased amount of label incorporation increases the fluorescent intensity of the probes, but also increases insolubility. A suitable probe intensity as assessed by acrylamide gel electrophoresis on an AB1373A DNA Sequencer photon multiplier tube voltage setting of 750-780 volts) includes visible, but non-saturating, fluorescent signal (100-4000 fluorescence units by the GeneScan software package, Applied Biosystems) on loading 0.5% of 488-labeled probes and 5% of 546-labeled probes.

Detection sensitivity also relies on adjusting the stoichiometry of chrormophore and template nucleic acid to maximize probe labeling. It was found as part of the present invention that, during cDNA synthesis by reverse transcription from template RNA, that the unlabeled dNTPs of the reaction mixture should include unlabeled dUTP, instead of dTTP typically required in standard procedures. The substitution of dUTP for dTTP improves efficiency of the mRNA labeling reaction because unlabeled dUTP competes less effectively than unlabeled dTTP for incorporation by reverse transcriptase, thereby increasing the number of chromophores incorporated into a probe.

As another method of improving detection sensitivity, the present invention contemplates use of ribonuclease (RNase), rather than commonly used alkali, to degrade the parent mRNA strands. It was discovered as part or the present invention that the omission of alkali in mRNA degradation was helpful because alkali substantially decreases the fluorescence emission of dye 488, one of the chromophores useful in the invention.

Preparation of Labeled DNA Probes:

While the present example discloses a method for preparing a DNA probe from RNA, it is also contemplated that DNA probes from RNA or DNA may be prepared based on the disclosure provided herein for related or alternative applications.

According to the invention, RNA strand extension was an initial step in cDNA probe synthesis. A basic technique for RNA strand extension is available from differential display reverse transcriptase PCR (DDRT-PCR). In that technique, total cellular RNA is primed for First strand reverse transcription with an anchoring primer composed of oligo-dT and any two of the four deoxynucleotides (DDRT-PCR; see, Liang and Pardee, Science. 257:967-971 (1992) and Russell, D. W. and Thigpen, A. E. , U.S. Pat. No. 5,861,248, issued Jan. 19, 1999). In one embodiment or the present invention, RNA strand extension uses an oligo-dTVN primer for extension by a reverse transcriptase, such as Moloney Murine Leukemia Virus reverse transcriptase (MMLV-RT) in the presence of dATP, dGTP, dCTP, dUTP, and chromophore-labeled dUTP, and other components as disclosed, infra. The present invention differs from DDRT-PCR, however, in that no amplification or only one round of amplification of the RNA or cDNA is performed. The methods disclosed herein improve detection sensitivity to such a surprising extent that detection and quantitation of gene expression may be performed on very small mRNA samples without the need for PCR-based or additional T7-based amplification or with only one round of linear amplification. As a result, the methods are rapid, convenient, and sensitive relative to existing methods.

Preparation of sDNA Probe

Detectably labeled sDNA probes were generated from 1 pg-10 ng total RNA. Because of the small amount of starting material, the present embodiment involves a single round of amplification prior to incorporation of chromophore as disclosed in the following procedure. The term “sDNA” refers to DNA generated from total cellular RNA by first and second strand cDNA synthesis, followed by one round (or optionally two rounds) of cRNA synthesis to amplify the nucleic acids sequences, followed by sDNA synthesis by reverse transcription of the cRNA in the presence of a detectably labeled dNTP.

First Strand Synthesis: Into each sample reaction vial was added: 10 ng purified total cellular RNA (isolated according to Example 1); 2 μg oligo-dTVN-T7 primer (oligo-dT refers to an oligomer of 18 dT residues complementary to poly-A tails of mRNA;V refers to nucleotides dA, dC, and dG; N refers to dA, dC, dG, and dT, and “T7” indicates that the oligo comprises the T7 promoter sequence, 5′-GAATTCTAATCGACTCACTATAGT₁₈-3′ (SEQ ID NO:1), at the 5′ end of the oligo); and 0.8 μl dNTP mix (500 μM each of dATP, dGTP, dCTP, and dTTP). The samples were heated to 65° C. for 3 min., cooled on ice, and left at room temperature for 10 min to anneal the primer to mRNA in the total cellular RNA mixture. To each sample was added 4 μl 5× reaction buffer (250 mM Tris-HCl, pH 8.3, 375 mM KCl, 15 mM MgCl₁); 0.5 μl RNase Block; 1 μl Superscript II; and 200 U Superscript reverse transcriptase (Life Technologies, Madison, Wis., USA) in a final volume of 20 μl. The samples were allowed to incubate at 42° C. for 1 hour to extend the first cDNA strand.

Second Strand Synthesis: To each sample vial from the First Strand Synthesis reaction, the following reagents were added: 91 μl DEPC water; 30 μl 5× reaction buffer, supra; 3 μl 10 mM dNTPs; 1 μl E. coli DNA ligase; 4 μl E. coli DNA polymerase; and 1 μl E. coli RNaseH. The samples were incubated at 16° C. for 2 hours.

In a related procedure, the reaction volume was reduced and the Klenow fragment of DNA polymerase I is used for an improved yield of double stranded DNA and subsequently sDNA probe. To each sample vial from the First Strand Synthesis reaction, the following reagents were added: 18.1 μl DEPC water; 10 μl 5× second strand buffer (Life Technologies); 1 μl 10 mM dNTPs; 0.3 μl E. coli DNA ligase (10 U/μl); 0.3 μl E. Coli DNA polymerase I Klenow fragment (50 U/μl); and 0.3 μl E. coli RNaseH (2 U/μl), for a total volume of 50 μl. The samples were incubated at 12° C. for 2 hours.

The resultant double stranded cDNA was partially purified by phenol:chloroform extraction. The cDNA was then precipitated by the addition of 85 μl 7.5 M ammonium acetate and 650 μl cold ethanol (approximately 0° C.) and 1 μl linear polyacrylamide, a nucleic acid carrier for precipitation (Ambion, Inc.). For the smaller volume reaction disclose above, the volumes were adjusted such that 29 μl 7.5 M ammonium acetate and 220 μl cold ethanol (approximately 0° C.) and 1 μl linear polyacrylamide were added. A cDNA pellet was collected, washed and dried by standard techniques.

Amplification by Transcription from cDNA: A single round of linear amplification is preferred when only small amounts of total cellular are available. Amplification is achieved by transcribing mRNA from the double stranded cDNA generated by first and second strand synthesis, supra. When only very small quantities of total cellular RNA were available from biological samples, (e.g. 1-20 pg of total RNA), the reaction was optionally followed as described, or the transcription reaction was allowed to continue overnight, or two rounds of linear amplification were performed. The following procedure describes a single round of linear amplification.

The double stranded cDNA was resuspended in 20 μl 1× T7 Transcription Reaction Buffer (Ambion, Austin, Tex., USA; T7 Megascript™ Kit, catalog no. 1337). To the resuspended cDNA were added the following components: 8 μl DEPC water; 2 μl each of 75 μM solutions of ATP, GTP, CTP, UTP; 2 μl 10× Buffer (Megascript™ Kit, Ambion, Inc.); 2 μl 10× T7 RNA polymerase. The samples were incubated at 37° C. for 5 hours. Overnight incubation under these conditions increased the yield. The reactions were stopped by the addition of 15 μl sodium acetate stop buffer (7.5 M sodium acetate), 115 μl DEPC water and extraction with phenol:chloroform. The nucleic acids were precipitated with an equal volume of isopropanol.

Incorporation of Fluorochromophore: Label may be incorporated into cDNA synthesized directly from mRNA present in total cellular RNA if 500 ng-5 μg or more is available. For direct cDNA synthesis from total cellular RNA, the following fluorochromophore incorporation procedure is useful. When less than 500 ng total cellular RNA was available, linear amplification as disclosed, supra, is preferred.

For probe synthesis after amplification, 5 ng-100 ng of cRNA pellet was suspended in 1× First Strand Reaction Buffer, supra. To the resuspended nucleic acids were added the following components: 1 μg random hexamers; 0.8 μl nucleotide mix (10 mM each dATP, dGTP, dCTP, and 7 mM dUTP); and DEPC water to bring the volume to 13.5 μl. The nucleic acids were denatured and the hexamers annealed by placing the samples at 65° C. for 3 min., chilling on ice, and then annealing at room temperature for 10 min. Optionally, from 100 ng to 10 μg random hexamers are added to the reaction.

Next, fluorochromophore was incorporated as follows: To each vial were added the following: 4 μl RNase Block; either dUTP-fluorophore (6-12 μM Alexa 546-dUTP or 25-40 μM Alexa 488-dUTP); and 1 μl MMLV reverse transcriptase (200 U). The reaction was incubated for 1 hour at 42° C. in the dark. The sDNA probes generated from control and test samples were labeled with different, detectably distinguishable chromophores. For example, the control probes were labeled with dye 546 and test probes were labeled with dye 488.

The parental RNA strands were removed from the sDNA probe mixture by RNase digestion according to the following protocol. Each reaction vial was heated to 95° C. for 1 min., followed by chilling on ice to denature the DNA and RNA strands. To each reaction vial, was added 1 μl diluted RNase (500 μg/ml diluted 1:50 in water; Boehringer-Mannheim). The RNase digestion was allowed to continue for 15 min. at 37° C. The reaction vials were then placed on ice until the next step could be performed.

As an aspect of the invention, the average sDNA probe length was controlled by the stoichiometry of random hexamer primer to cRNA such that the average probe length was 0.5-2 kb. As the ratio or random primers to cRNA increased, the average probe length (related to average probe molecular weight) decreased.

Preparation of Labeled cDNA Probe Directly from Total Cellular RNA In another aspect, the invention involves a method of preparing labeled cDNA probes directly from total cellular RNA by incorporating detectably labeled dNTPs in the reaction mixture for first strand synthesis according to the first strand synthesis procedure disclosed, supra.

According to this method, first strand cDNA synthesis with direct label incorporation was performed as follows. Into each sample reaction vial was added: 1-10 μg purified total cellular RNA (isolated according to Example 1); 2 μg oligo-dTVN-T7 primer (oligo-dT refers to an oligomer of 18 dT residues complementary to poly-A tails of mRNA;V refers to nucleotides dA, dC, and dG; N refers to dA, dC, dG, and dT, and “T7” indicates that the oligo comprises the T7 promoter sequence. 5′-GAATTCTAATCGACTCACTATAGT₁₈-3′ (SEQ ID NO:1), at the 5′ end of the oligo); and 0.8 μl dNTP mix (500 μM each of dATP, dGTP, dCTP, and dTTP). The samples were heated to 65 C for 3 min., cooled on ice, and left at room temperature for 10 min to anneal the primer to mRNA in the total cellular RNA mixture. To each sample was added 4 μl 5× reaction buffer (250 mM Tris-HCl. pH 8.3, 375 mM KCl, 15 mM MgCl₂); 0.5 μl RNase Block (Stratagene); 1 μl Superscript II; and 200 U Superscript reverse transcriptase (Life Technologies. Madison, Wis., USA) in a final volume of 20 μl. The samples were allowed to incubate at 42° C. for 1 hour to extend the first cDNA strand.

The average cDNA probe length was next adjusted with limited Dnase digestion. The cDNA reaction volume in each vial was adjusted to 50 μl with 10 mM MgCl2 and chilled on ice. A dilute DNase I solution was prepared comprising 1 part DNase I (10,000 U/ml; Boehringer-Mannheim) in 5000 parts 20 mM Tris buffer, pH 8.0. The final dilution of DNase I was approximately 2 U/ml. A 2 μl aliquot of diluted DNase I (2 U/ml) was added to each vial containing cDNA probe labeled with dye 546, and a 4 μl aliquot of diluted DNase I was added to vials containing cDNA probe labeled with dye 488. The DNase conditions may be varied as necessary to adjust for different chromophores and input cDNA. The vials were incubated at 12° C. for 30 min. Next 5 μl 250 mM EDTA pH 8.0 was added to each vial. DNase I was inactivated by heating each vial to 65° C. for 15 min. The labeled cDNA probe was separated from the proteins by standard phenol:chloroform extraction followed by purification of the aqueous layer over a G50 spin column (Pharmacia). To each aqueous eluate from the spin columns was added a 3 μl aliquot of a 10×SSC solution. The cDNA probe pellet was dried and resuspended in a 6 μl aliquot of 50:50 formamide:water solution for at least 3-4 hours at room temperature in the dark. Once a flurochromophore is incorporated into a probe, the probe is preferably kept in the dark at 0° C. or below until ready to use. The resuspended labeled cDNA probe is useful for hybridization to microarrays as disclosed herein.

Preparation of Labeled sDNA Probe Directly from First Strand cDNA In another aspect, the invention involves a method of preparing labeled sDNA probes directly from cDNA without intermediate cRNA synthesis (without amplification). The probes are prepared by second strand sDNA synthesis with simultaneous incorporation of label. Average probe length is controlled by the use of random primers in the final labeling step. The method is similar to the method for preparation of labeled cDNA probes with the following modifications. The probe labeling involves double stranded cDNA preparation as disclosed, supra, followed by labeling of sense strand DNA (sDNA) using fluorescent deoxyribonucleotides and random primers. The unlabeled first strand DNA is synthesized using a biotin-labeled primer and can be removed, to avoid competition in hybridization, using streptavidin. A non-limiting example of the method follows.

RNA isolation from samples: RNA was prepared from frozen tissue, samples isolated by laser capture microdissection (LCM), or from tissue stored in RNAlater reagent (Ambion, Austin, Tex., or Qiagen, Valencia, Calif.). Samples were homogenized with a rotor-stator tissue homogenizer (IKA Labortechnik, Staufen, Germany, or Brinkman Instruments, Westbury, N.Y.) in RLT buffer according to the RNeasy Mini or Midi RNA purification kits (Qiagen, Valencia, Calif.). Purified RNA was quantified by measuring optical absorption at 260 nm in a UV spectrophotometer (Shimadzu, Pleasanton, Calif.). For RNA purified from small amounts of tissue ( <1 μg of tissue, or LCM tissue sample) the RiboGreen RNA quantitation assay (Molecular Probes, Eugene, Oreg.) was used with a fluorescence microplate reader (Molecular Devices, Sunnyvale, Calif.).

First Strand cDNA Synthesis: First strand cDNA was synthesized from 0.5-5 μg of total RNA using Superscript reverse transcriptase as described by the manufacturer (Life Technologies, Rockville, Md.) using 5′-biotin labeled (dT)₁₈VN, where V=G, A, or C and N=G, A, T, or C. RNA was then digested in 10 ng of Dnase-free RNase A (Roche Molecular Biochemicals, Indianapolis, Ind.) 37° C. for 15 minutes. The reaction was extracted with water saturated phenol:chloroform:isoamylalcohol (49:49:2). Linear acrylamide (Ambion, Austin. Tex.) was added to a final concentration of 18 ng/ml. One-tenth volume of 3 M sodium acetate pH 4.8 was added and cDNA was precipitated by the addition of an equal volume of ice cold isopropanol. Samples were incubated at −20° C. for 20 minutes, centrifuged at 14,000 rpm for 20 minutes at 4° C., and the supernatant was aspirated from the clear pellet which was vacuum dried.

Second Strand cDNA Synthesis of Incorporation of Fluorochrome: Second strand cDNA was synthesized in 20 μl reaction using 2 Units of the Klenow fragment of DNA polymerase I (Life Technologies, Rockville, Md.), 1 to 50 μg of p(dN)₆ (Life Technologies, Rockville, Md.) or other random sequence oligonucleotide of 7 to 9 bases, 100 μM each of dGTP, dCTP, and dATP, and a combination of dTTP and Alexa488-dUTP (Molecular Probes, Eugene, Oreg.) to a final concentration of 100 mM. The dTTP to Alexa488-dUTP ratio may vary from 100:1 dTTP to Alexa488-dUTP to 100% Alexa488-dUTP. Alexa 546-dUTP and Alexa 594-dUTP may also be used with this protocol. NaCl may be added in addition to the standard working concentration of 50 mM, increasing in concentration up to approximately 150 mM. The reaction included reaction buffer components supplied by the enzyme supplier (Life Technologies, Rockville, Md.). Reactions were initiated by first heating the reaction mixture to 95° C. for 5 minutes, then quickly chilling it on ice, followed by the addition of Klenow enzyme. The reaction was incubated at temperatures ranging from 12° C. to 37° C. for 1 to 18 hours. Reactions were stopped by addition of EDTA to 25 mM, heated at 95° C. for 5 minutes and quickly chilled on ice. The biotin-tagged (−) strand cDNA is separated from random-primed (+)-strand labeled cDNA using streptavidin-para magnetic particles (SA-PMP) (Promega, Madison, Wis.). SA-PMPs were prepared by washing 3 times in 0.5×SSC and once in 10 mM Tris, 1 mM EDTA, pH 7.5. The labeled cDNA reaction was incubated with the SA-PMPs for 10 minutes at room temperature, and the supernatant was removed from the SA-PMPs on a magnetic stand. The resulting labelled (+) strand cDNA was extracted with water saturated phenol:chloroform:isoamylalcohol (49:49:2), purified over a G-50 spin column (Pharmacia), and vacuum dried before using in a hybridization reaction.

Preparation of Labeled cRNA probes: Preparation of labeled cRNA probes is preformed, according to the invention, by direct incorporation of fluorochromophore-labeled ribonucleotides into cRNA followed by adjustment of average probe length. The method is similar to the method for preparation of labeled cDNA probes with the following modifications. The cRNA probe synthesis begins from the step of double stranded cDNA preparation as disclosed, supra.

Double stranded cDNA prepared was resuspended in 20 μl 1× T7 Transcription Reaction Buffer (Ambion, Austin, Tex., USA; T7 Megascript™ Kit, catalog no. 1337). To the resuspended cDNA were added the following components: 8 μl DEPC water; 2 μl each of 3.75 mM solutions of ATP, GTP, CTP, UTP; 2 μl 10× Buffer (Megascrip™ Kit, Ambion, Inc.); 2 μl 10× T7 RNA polymerase. To each vial were added the following: 4 μl RNase Block; either UTP-fluorophore (60 μM Alexa 546-UTP (preferably in the range of 30-120 μM, inclusive) or 300 μM Alexa 488-UTP (preferably in the range of 200-400 μM, inclusive)). The samples were incubated at 37° C. for 5 hours, or overnight for further improvements in yield. The reactions were stopped by the addition of 15 μl sodium acetate stop buffer (7.5 M sodium acetate), 115 μl DEPC water and extraction with phenol:chloroform. The nucleic acids were precipitated with an equal volume of isopropanol. Using this, procedure, the cRNA probes were generated from control and test samples and were labeled with different, detectably distinguishable chromophores. For example, the control probes were labeled with dye 546 and test probes were labeled with dye 488.

The preferred average cRNA probe length was from 0.5 kb to and including 3 kb. The average probe length and labeling density of the cRNA probes was estimated by observing the probes on a sequencing gel such as an ABI 373A gel (Applied Biosystems, USA). The labeling density was estimated according to an observed correlation between an increase in labeling density and the ratio of labeled to unlabeled cRNA probe.

If it was determined that the average length should be reduced, the labeled cRNA probe length was adjusted by resuspending the precipitated, labeled cRNA probes in 40 mM tris-acetate, pH 8.1. 100 mM potassium acetate, 30 mM magnesium acetate. The resuspended, labeled cRNA probes were incubated at 70° C. for 10 min. Optionally, mild RNase digestion may be used to decrease the average length of the cRNA probes. It is understood that reaction conditions may vary and are readily adjusted depending on the beginning average probe length and label density. Once a flurochromophore is incorporated into a probe, the probe is preferably kept in the dark at 0° C. or below until ready to use. The resuspended labeled cDNA probe is useful ror hybridization to microarrays according to the invention.

The preferred average DNA probe, sDNA probe, or cRNA probe length was from 0.5 kb to and including 3 kb, preferably from 0.5 kb to and including about 2 kb. The average probe length and labeling density of the sDNA probes was estimated by observing the probes on a sequencing gel such as an ABI 373A gel (Applied Biosystems, USA). The labeling density was estimated according to an observed correlation between an increase in labeling density and the ratio of labeled to unlabeled probe. The stoichiometry of random hexamers to cRNA is the preferred method for controlling the average sDNA probe length. The average length of cDNA probes is preferably adjusted by mild Dnase digestion as disclosed herein.

Design of Controls for Microarray Analysis: In the present examples, carcinomas, cancers of epithelial tissue, were studied for gene expression relative to nonconcerous tissue. For this purpose, matched noncancerous tissue (i.e. “normal” tissue) is of limited availability. A “universal” epithelial control was prepared by pooling noncancerous tissues of epithelial origin, including liver, kidney, and lung. RNA isolated from the pooled tissue represents a mixture of expressed gene products from these tissues. The pooled control referred to hereinafter as the “control” sample, was an effective control for relative gene expression studies of tumor tissue and tumorigenic cell lines. Microarray hybridization experiments using the pooled control samples generated a linear plot in a 2-color analysis as disclosed herein. Because the test and control samples have many genes expressed at similar quantitative levels, a plot of intensity data for all of the target molecules that formed complexes with the control and test probes yielded a linear clustering of the data. The slope of the line fitted to these data in a 2-color analysis was then used to normalize the ratios of test to control within each experiment. The normalized ratios from various experiments were then compared and used to identify clustering of gene expression, and genes differentially expressed in diseased tissue versus normal tissue across many different tissue samples. Thus, the pooled “universal” control sample not only allowed effective relative gene expression determinations in a simple 2-sample comparison, it also allowed multi-sample comparisons across several experiments.

### Example 3

### Microarray Slide Preparation

Activated glass slides used for attachment of target molecule polynucleotides in nucleic acid microarray preparation are commonly treated with polylysine (see, for example, U.S. Pat. No. 5,807,522) or organosilane (See, for example, WO 01/06011; WO 00/40593; U.S. Pat. No. 5,760,130; and Weiler et al., Nucleic Acids Research 25(14):2792-2799 (1997)). For the purposes of the present invention, organosilane-based treatment of the glass slide was preferred because it allowed specific nucleic acid sequence end attachment via a covalently attached primary amine on the nucleic acid as disclosed herein. Such specific attachment is advantageous for specific positioning of nucleic acid sequences on a microarray slide, thereby ensuring attachment of the nucleic acid while rendering it free to hybridize efficiently with complementary sequences in a probe.

It was discovered as part of the present invention that even unmodified nucleic acids (such as target DNA) can attach to a glass slide treated with 3-aminopropyltriethoxysilane (APS) followed by attachment of phenylene diisothiocyanate, suggesting that the nucleic acids may also be attaching a functional group on unmodified DNA (for example, at the 5′ end of an unmodified promer used to amplify nucleic acids for arraying by PCR, or amines on unmodified DNA bases). Thus, the invention involves the attachment of unmodified polynucleotides to an activated microarray slide of the invention.

The present inventors also discovered that the solvent used for silane treatment of glass slides has a marked affect on the fluorescent background observed in microarray analysis. Acetone, the commonly used solvent for dissolving silane during glass slide treatment, caused a high and/or non-uniform fluorescent background during imaging. Methanol is occasionally used as a solvent for silanization (see, for example, <http://sgio2.biotec.psu.edu/protocols/silanize.html> (last visited Mar. 13, 2001). Methanol is disadvantageous because water present in methanol quenches the silanizing reaction and limits efficient coating of the a glass microarray slide. For examples of other procedures for silanization in solvents other than toluene, see, WO 01/06011; WO 00/40593; U.S. Pat. No. 5,760,130; and Weiler et al., (1997), supra). Because efficient silanization and low background is preferred for maximum signal-to-noise ratio and highest sensitivity, an alternative solvent was sought. Toluene was found to be a superior solvent for silane treatment because longer glass treatment could be used to ensure optimal coating while avoiding high fluorescent background. Acetone is still useful in the glass slide treatment method of the invention, but its use is preferably confined to drying steps where contact with acetone is of relatively short duration.

Preparation of Activated Microarray Slides:

According to the method of the invention, glass slides were treated using the following protocol to prepare them for use in making nucleic acid microarray slides.

Cleaning Glass Slides: Glass microscope slides (standard size) were used for the present experiment. Throughout the procedure, the slides were handled with solvent-proof gloved hands. Thirty slides were loaded onto a clean metal rack and the rack was lowered in a clean ultrasonic cleaner chamber filled with 1% Liquinox™ (Alconox, NY N.Y.) in highly purified water, such as by reverse osmosis (designated “SQ water”; MilliQ™ System, Millipore Corp., Bedford, Mass.) The Liquinox solution was heated to approximately 50 C in the ultrasonic cleaner prior to immersing the slides. The slides were cleaned ultrasonically for 30 min. at 50 C. The same solution of Liquinox may be used to clean approximately 4 batches of 30 slides per batch. After cleaning, the slides were transferred to a plastic container filled with deionized water. The plastic container is preferably used only for rinsing cleaned slides to avoid contamination or the slides with extraneous material. The slides were rinsed three times with running deionized water and then placed on a shaker. The rinsing and shaking steps were repeated six times to ensure thorough rinsing. The final rinse was with SQ water. The slides were stored in SQ water until use.

In a preferred cleaning method according to the invention, slides were loaded in glass racks, 20 slides per rack, and cleaned in a clean ultrsonic cleaner chamber filled with 3% GLPC-Acid™ in highly purified water, such as by reverse osmosis (designated “SQ water,” MilliQ™ System, Millipore Corp., Bedford, Mass.), for 20 minutes at 65° C. After cleaning, the slides were rinsed thoroughly with deionized water. The slides were then placed in an ultrasonic cleaner chamber containing 0.5% sodium hydroxide, 50% ethanol and treated for 10 minutes at 65° C. The slides were rinsed very thoroughly with deionized water and the final rinse was with SQ water. The slides were stored in SQ water until use the next day.

All subsequent procedures for slide silanization were performed in a well-ventilated fume hood.

Drying Slides: The clean slides were transferred in the metal rack to a glass chamber. The slides were covered with acetone, shaken briefly, and removed from the acetone. The slides are allowed to drain and then dry in the fume hood. The slides were protected from exposure to dusty air that may be drawn into the fume hood by placing the slides behind the glass chamber in the hood and/or placing them back in the glass chamber after the acetone is removed and the chamber allowed to dry. The slides remained in the glass chamber until dry and free of water or acetone because these solvents are problematic: water interferes with silanization and acetone causes high fluorescent background.

Silanizing Glass Slides: Screw-cap Coplin staining jars were cleaned and dried completely. Preferably drying is performed in a drying oven. The clean glass slides were transferred into the dry staining jars using gloved hands and forceps by handling the slides only at the corners. A solution of 10% 3-aminopropyltriethoxysilane in toluene (substantially water-free as purchased from Burdick and Jackson) was prepared by adding the silane to the toluene and swirling to mix. Immediately after mixing, the silane solution was poured over the slides in each jar. Approximately 550 ml silane solution filled 6 jars. The jars were quickly covered with the screw-caps such that air and moisture were excluded from each jar to avoid precipitation of silane polymers on the slides. The slides were silanized overnight.

In a preferred method of silanizing glass slides according to the invention, a solution of 2% 3-aminopropyltriethoxysilane in toluene (reagent grade) was prepared by adding the silane to the toluene and swirling to mix. Immediately after mixing, the silane solution was poured over the slides in each glass chamber. The glass chambers were completely filled to the top edge and a lid was placed on top. The slides were silanized 14 hours at room temperature. Preferably, the 2% silanizing procedure is applied to glass slides cleaned in 0.5% NaOH, 5% ethanol (as disclosed supra).

Washing Silanized Slides: Following silanization, slides were washed by the following procedure. Glass washing chambers containing slide racks were filled with toluene. A glass chamber that holds 10 slides is filled with 250 ml toluene and a chamber that holds 20 slides requires approximately 400 ml toluene. The silanized slides were transferred from the silanization solution to racks submerged in toluene in the glass chambers using forceps to handle the slides only at the corners and without allowing the slides to dry during the transfer. In another washing procedure and at the end of the silanization period, the silanization chamber was emptied and filled with toluene such that the rack of slides was covered.

At this point in either of these washing procedures, a clean glass lid was placed on top of the chamber and the chamber was agitated for 2-6 min. The glass lid was then removed and inverted on the counter top to provide a platform on which the rack of washed slides were placed. The toluene was discarded from the chamber. The toluene wash was repeated twice. The slides were not allowed to dry during the wash procedures.

The third toluene wash was discarded, the chamber drained, and methanol was added to the chamber. Slides were submerged in the methanol and agitated for approximately 5 min. Slides were washed twice with agitation in SQ water for 5 min per wash. The slides were then washed twice in methanol ror approximately 4-5 min. with agitation.

As the final wash step, the slides were rinsed with acetone for 1 min to speed drying. The slides were allowed to dry completely in the fume hood. The slides were protected from dust by placing them in the empty glass chambers used for the wash steps. At this stage, the slides were stable for approximately one hour. In another method following the acetone rinse, slides were rinsed in dimethylformamide (DMF). The DMF was then drained from the chamber. After these wash procedures, the slides were prepared for attachment of a bifunctional linker reagent.

PDITC Attachment to Silanized Slides: The surface of the silanized slides was next cross-linked using 1,4-phenylene diisothiocyanate (PDITC), a bifunctional cross-linking agent (see Greg T. Hermanson, Bioconjugate Techniques, Academic Press (1996)) capable of reacting with silane on the glass slide at one end, and with amino-derivatized microarray DNA at the other end. Microarray DNA is thus firmly attached to the glass surface. The PDITC linkage is water sensitive, however. As a result, the slides must remain free of water until after attachment of the target molecule, such as a target polynucleotide.

The PDITC solution was prepared as follows. To a solution of 90% dimethyl formamide (DMF) and 10% pyridine, an appropriate amount of solid PDITC was added to provide a 0.20-0.25% PDITC concentration and, as expected, the solution was yellow. Due its reactivity, solid PDITC was handled quickly and stored under argon.

The PDITC solution was poured over the silanized slides, still in the glass chambers in the fume hood, and the chambers were completely filled. The glass lids were placed on the chambers and each chamber was covered with foil to block exposure to light. The slides were incubated in PDITC for 2 hours.

Following incubation, the PDITC solution was removed. DMF was added to the chambers and the slides were agitated for 3-5 min. The DMF wash was repeated twice more with agitation for approximately 5 min per wash.

The slides were then washed twice with methanol for 3-5 min. with agitation. The slides were not left in methanol for longer than 5 min. because traces of water in methanol could react with the PDITC. The slides were washed 3 times with agitation in acetone for 3-5 min. per wash. The slides were then dried completely in the fume hood, protected from dust. The PDITC-treated slides were then stored in a dry cabinet. The slides are stable under these conditions for at least 3 months.

### Example 4

### Attaching Target Molecules to an Activated Microarray Slide

It is understood that microarrays may be prepared by the user or purchased commercially. Descriptions or microarrays on glass slides are available in, for example, U.S. Pat. No. 6,040,138. Generally, a DNA microarray on a glass slide contains at least 100, preferably at least 400 or more DNA samples of at least partially known sequence in known locations on the slide at a density of at least 60 oligonucleotide sequences per square centimeter. The microarray sequences may be oligonucleotides of 5-100 nucleotides in length, or the sequences may be polynucleotides from 50 nt to 10 kb in length, or they may be full length gene sequences. A sufficient portion of each sequence must be known so that it is distinguishable from the other sequences, and it must be long enough to hybridize to a labeled probe under the conditions used.

Preparation of Target Nucleic Acid Sequences:

In this example, nucleic acid sequences of interest (“target sequences,” “target polynucleotides,” or “targets”) were generated from full length or partial cDNA clones. Optionally, a target was cloned into a vector for ease of manipulation. The target sequence (i.e. non-vector nucleic acid sequence of interest) was amplified by the polymerase chain reaction (PCR) using “Klentaq GC melt” DNA polymerase (Clontech). This enzyme provided a high success rate of amplifying DNA inserts, with uniform yields, across a range or templates that varied in both length (0.25-4 kb) and nucleotide composition.

As disclosed herein, an unmodified polynucleotide attaches directly to an activated glass slide prepared by silanizing with an organosilane in toluene, followed by reaction with a multifunctional linker reagent that is capable of reacting with the unmodified polynucleotide. As the examples herein disclose, the organosilane may be APS and the multifunctional linker reagent may be PDITC.

Alternatively, the target molecule may be modified by incorporation of a reactive group in the target molecule, which reactive group is reactive with a functionality on the multifunctional linker reagent of the activated microarray slide of the invention. According to this alternative method of the invention and simultaneous with amplification of target sequences, the amplified targets were modified to comprise a linker for covalent attachment to a solid support of a microarray. To accomplish simultaneous amplification and modification, PCR primers had at least two features. First, the PCR primers were complementary to the vector sequences into which the target DNA was inserted, thereby ensuring amplification of the complete target sequence. Further, the primer from which the modified single strand target DNA would be generated comprised a reactive moiety: a primary amine linked to the primer's 5′ end via a linker, preferably an alkyl linker, such as a —(CH₂)₆— linker. For the purpose of this example, such a primer had the following general structure: 5′ NH₂—(CH₂)₆-dNx 3′, where NH₂ is a primary amine group, (CH₂)₆ is a methylene linker, and dNx is a nucleotide sequence, preferably an oligonucleotide sequence (DNA in this example), complementary to a portion of the vector into which the target DNA was inserted (primers were synthesized at Genentech, Inc. So. San Francisco, Calif., USA). Preferably, the dNx sequence hybridizes to a vector sequence near the target insert such that enzyme-driven elongation of the primer into the target sequence using two vector-specific primers that flank the target sequences. Nucleic acid synthesis resulted in formation of a double stranded nucleic acid sequence complementary to the target sequence, wherein the complementary region is at least 10 nucleotide bases is length. Thus, following PCR amplification, each target sequence comprised a primary amino group on its 5′ end, which amino group was capable of reacting with a reactive group on an activated slide. For example, as disclosed herein by a non-limiting example, a primary amine incorporated into a polynucleotide allows immobilization of the polynucleotide on an activated glass slide. According to the invention, a glass slide is activated by silanizing in toluene with a organosilane that is then reacted with a multifunctional linker reagent. The multifunctional linker reagent is reactive with both the organosilane on the surface of the glass and with a primary amine of a modified polynucleotide as disclosed above.

Prior to immobilization on an activated slide, PCR-amplified double stranded target DNA sequences were purified using glass-fiber filters (Qiagen, Valencia, Calif.). A portion of the purified sequences was analyzed by agarose gel electrophoresis for correct molecular weight, purity (e.g. a single band representing a single product and not a mixture of clones or genes) and approximate yield of DNA (estimated by fluorescent staining with ethidium bromide following standard procedures).

The primary amine-modified target sequences were resuspended in an arraying buffer (500 mM sodium chloride, 100 mM sodium borate, pH 9.3, which promotes reaction between the primary amine of the modified target DNA and the PDITC-derivatized, silanized glass surface, resulting in covalent attachment of the target DNA to the glass slide. The slides were ready for use according to the invention, increased attachment and improved detection intensity was achieved when the slides were allowed to remain at ambient temperature and humidity in the dark overnight, such as for approximately 10-16 hours. A concentration of modified target sequence of at least 0.1 μg/μl provided successful covalent attachment to the activated glass slides, good spot morphology, and a sufficient number of covalently attached target sequences such that they were in excess relative to fluorescently labeled cDNA probes applied during subsequent hybridization reactions. This permitted quantitative measurement of the absolute fluorescent signals obtained after probe hybridization.

In this example, a two-step protocol was used to attach nucleic acids, such as gene sequences, to the silanized, PDITC-treated glass slides prepared according to the present invention. It is understood that fewer steps or more steps may be used as long as any silanizing step is performed in toluene in the absence of acetone or an an alcohol according to the present invention.

As disclosed, supra, the slides were first silanized with 3-aminopropyltriethoxysilane (APS) in toluene. The slides were then treated with PDITC (1,4-phenylene diisothiocyanate), a multifunctional linker reagent which contains two amine-reactive isothiocyanate groups. One of the isothiocyanate groups reacts with the amine group or the organosilane. The second isothiocyanate group is available to react with a primary amine present on the 5′ ends of the modified target DNA (see Example 1), thereby providing the means of attaching the target DNA to the glass slide during spotting of the DNA onto the microarray. After attaching the modified target sequences, the slides were washed once in water containing 0.2% SDS, then washed three times in SQ water, and finally dipped in ethanol and dried. Slides cleaned, silanized, and PDITC-treated according to the method of the invention were superior substrates for nucleic acid microarrays because fluorescent background was minimized, and hybridization was enhanced by minimizing over-attachment of the arrayed target DNA, thereby providing a surprising increase in detection levels over previous methods.

Microarrays Comprising Single Stranded Target Oligonucleotides

Improved microarrays comprising single stranded target oligonucleotides are encompassed by the present invention. A non-limiting example of the arrays and a method of making them follows.

Single stranded target DNA for array fabrication was synthesized by standard solid-phase methods with a 3′-C7 amino linker (Glenn Research, Sterling, Va.) with or without hexethyleneglycol spacers (S18) (Glenn Research, Sterling, Va.) incorporated between the 3′-end of the synthetic DNA and the C7 linker.

Single stranded DNA molecules, such as chemically synthesized target oligonucleotides of approximately 50 to 100 nucleotides in length were immobilized onto activated microarray slides of the invention (e.g. aminosilane in toluene/PDITC-treated glass) by standard microarray printing techniques. The printing solution comprised oligonucleotides at a concentration of up to 10 μM in 0.1 M borate, 0.5 M NaCl, pH 9.3. The slides were dried overnight at 20° C. and ambient room humidity. It was discovered as part of the present invention that drying overnight generated microarrays capable of providing an increased fluorescent signal when hybridized with polynucleotide probes of the invention.

Improved detection signal was demonstrated by hybridizing a complementary fluorescein-labeled 100 mer single strand DNA fragment to the single stranded target oligonucleotide DNA arrays as disclosed, supra, revealed that hybridization signal intensity was dependent on immobilized DNA length, with longer DNA strands providing a stronger signal. In addition, varying the number of S18 repeats from 0 to 6 revealed increasing signal intensity with increasing tether length. The combination of a 100 nucleotide single stranded target DNA molecule with 6-S18 repeats and a C7 amino linker provided highest hybridization signal intensity. Accordingly, microarrays of the invention comprising single stranded target DNA oligonucleotides are improved when the distance of the oligonucleotide from the solid surface and DNA chain length are increased.

Spotting Target Molecules onto Activated Slide

Target DNA (modified or unmodified) in 5-10 μl 100 mM sodium borate pH 9.3, 500 mM sodium chloride, in 384 well plates, was used for arraying the target DNA onto activated microarray slides of the invention. Arraying, (also termed printing or spotting) target molecules on an array slide, was performed using an automated microarraying device equipped with a printing pin having a 80 micron internal width (TeleChem International. Inc., model no. CMP2, “Chipmaker 2 Microspotting Pins”). Approximately 0.5-1 nl of target solution was deposited at each array element (spot or location) using the printing pin. Spot size was regulated at 100-140 microns in diameter due to the tip diameter and the nature of the surface generated on the slides prepared according to the invention. Due to the buffer used for printing and the reactivity of the slides of the invention nucleic acid molecules attach rapidly with no further manipulations. It was discovered as part of the invention that leaving the printed slides at ambient conditions overnight increased attachment of target DNA to the microarray slides in some cases.

Following spotting, slides were placed in glass racks and washed in 0.2% SDS, followed by three washes in SQ water, followed by an ethanol rinse. This washing procedure removes unattached target DNA and modifies unreacted thiocyanate functionalities. Printed, washed slides were allowed to dry and stored in slide boxes in the dark under ambient conditions.

### Example 5

### Hybridization Method for Microarray Analysis

The miroarray hybridization method disclosed herein allows enhanced nucleic interaction for improved hybridization and higher signal-to-noise ratio for more sensitive detection. Greater sensitivity is useful when samples, such as tissue samples, are small and limited.

According to the present invention, formamide and/or dimethylsulfoxide are used to suspend labeled oligonucleotide probes because the fluorescently labeled DNA probe is more soluble in these polar ogranic solvents. Preferably, the amount of polar organic solvent in the hybridization solution is not more than 50%, 40%, 30%, 25%, or 20%. According to the invention, the proportion of DMSO is from 0% to and including 50%, from 0 to and including 40%, from 0 to and including 30%, from 0 to and including 25%, and from 0 to and including 20%. Similarly, the proportion of formamide is from 0% to and including 50%, from 0 to and including 40%, from 0 to and including 30%, from 0 to and including 25%, and from 0 to and including 20%. Thus, according to the invention, the total amount of polar organic solvent (either DMSO or formamide) does not exceed 50%, for example, which the relative proportion of DMSO to formamide is varied from such that the sum of the proportions of these organic solvents does not exceed 50%, in this example.

In addition, it was discovered by the present inventors that the omission of detergent, sodium dodecyl sulfate (SDS) for example, from the hybridization conditions improved detection. It was discovered that SDS caused the formation of colloidal ;complexes with the fluoorescently labeled DNA probe, causing the probes to precipitate out of solution, limiting detection, and/or causing unwanted detection variability, and/or very high non-specific fluorescent background. The absence of the solid surface wetting capabilities of SDS were overcome by the use of formamide in the hybridization and the glass surface treatment disclosed herein.

The microarray hybridization method of the invention comprises the following protocol. Before application of the probe, the microarray was denatured by placing it at 95° C. for 2 min. The microarray was then submerged in cold ethanol (approximately 20° C.) to quickly cool it to room temperature and to maintain the denatured state of the sequences in the array. Probes were resuspended in a Final concentration of 5×SSC, 50% formamide. The resuspension was allowed to continue For at least 3 hours and up to overnight (e.g. approximately 10-16 hours in the dark. The control and test probes were pooled, heated to 95-100° C. for 45 seconds, and, while hot, applied as 10 μl aliquots to the surface of the denatured microarray slide, which was on a slide warmer at approximately 50° C. Following application of the probes, a clean glass coverslip was carefully placed over the array to cover it. The covered microarray slide was placed in a hybridization chamber at 37° C. overnight. The hybridization chamber may be any vapor-tight, chemically inert container. For example, the hybridization chamber used in the present example was a plastic container having a vapor-tight plastic lid into which were placed absorbent material, such as paper towels, wet with 50:50 Formamide:water. The interior or the chamber was allowed to equilibrate at 37 C for at least 30 min prior to use.

Hybridization in Alkylammonium Salt, DMSO, and Formamide

It was discovered as part of the invention that alkylammonium salts, dimethylsulfoxide (DMSO), and formamide in the microarray hybridization buffer improved detection sensitivity.

Alexa-dye (Molecular Probes, Eugene, Oreg.) labelled cDNA probes, either + or − strand, may be hybridized to cDNA or oligonucleotide arrays in 2.4 M TEACl (Alfa Aesar, Ward Hill, Mass.) or 3.0 M TEACl (Sigima. St Louis, Mo.) with 50 mM Tris (Sigma). 2 mM EDTA (Sigma) at pH 8.0. The polar solvents formamide (Life Technologies, Rockville, Md.) and dimethylsulfoxide (DMSO) (Sigma) were also included in the array hybridization solution in varying proportions up to a final total concentration of DMSO and formamide of 25% (v/v). In other words, formamide and DMSO concentrations may vary from 25% (v/v) fonnamide and 0% (v/v) DMSO to 0% (v/v) formamide and 25% (v/v) DMSO, for example 20% (v/v) formamide, 5% (v/v) DMSO. It was found as part of the present invention that hybridization or a fluorescently labeled polynucleotide to an oligonucleotide array as disclosed herein was improved when TEACI and DMSO were in the hybridization buffer.

For example, signal intensity using a first hybridization buffer (Buffer 1) comprising 50% (v/v) formamide, 5×SSC buffer was compared to a second hybridization buffer (Buffer 2) comprising 2.4 M TEACl, 50 mM Tris, 2 mM EDTA, pH 8.0, with 20% formamide/5% (v/v) DMSO. Separate (−) strand labeled cDNA probe mixture were prepared with Alexa488-dUTP or Alexa546-dUTP (Molecular Probes, Eugene, Oreg.) by second strand synthesis with simultaneous label incorporation as disclosed herein. Each labeled probe mixture was divided into equal aliquots and vacuum dried. Samples were resuspended in either 50% (v/v) formamide, 5×SSC buffer (Buffer 1) or 2.4 M TEACl, 50 mM Tris, 2 mM EDTA, pH 8.0, with 20% formamide/5% (v/v) DMSO (Buffer 2). 488 and 456 labeled probes were pooled and each different probe pool was hybridized to one of a microarray duplicate. The results demonstrated fluorescence signal intensity was improved for each label in Buffer 2 relative to Buffer 1 as a result of the addition of TEACl and DMSO. The hybridization signal found with 2.4 M TEACl, 50 mM Tris, 2 mM EDTA, pH 8.0, with 20% formamide/5% (v/v) DMSO was increased 3-5 fold over the signal obtained in hybridization buffer lacking TEACl and DMSO.

After hybridization, the microarray slides were taken from the chamber. The coverslip was carefully removed and the slides were washed in 2×SSC, 0.2% SDS for 2-5 min., followed by a wash with 0.2×SSC, 0.2% SDS for 2-5 minutes. The slide was covered with a new. clean coverslip to keep the array region wet with the last wash solution while imaging of the hybridized array was performed. Imaging the slides while wet avoids quenching of the chromophores, thus improving both the absolute signal and the quantitative nature of the signal. The top and bottom of the slide were otherwise kept dry. Imaging did not bleach the chromophores and the hybridized microarrays may be stored in the dark for re-imaging for at least 60 days.

### Example 6

### Detection Method

Means for detecting the labeled hybridized probes are well known to those skilled in the art. In the present example where fluorescently labeled probes were applied to densely arrayed nucleic acid sequences, detection is preferably performed by fluorescence imaging. Alternatively, a CCD camera imaging system was used. For, example, excitation of the chromophores using fluorescence spectroscopy occurs by exposing the hybridized slide to a fluorescent laser or other light source through a filter specific for the desired excitation wavelength. Fluorescent emission was detected at the discrete emission wavelength for each chromophore. The relative emission of test and control probes was analyzed according to the chromophore incorporated into each probe type and the specific microarray member to which a probe hybridized. The analysis provided quantitative information on the relative expression of the genes in diseased tissue. Where automated detection and analysis are desired, an automated system for detecting and quantifying relative hybridization is found, for example, in U.S. Pat. No. 5,143,854, which detection procedures are herein incorporated by reference.

Microarray slides hybridized with a mixture of test and control probes were viewed using an imaging device configured for fluorescence excitation at 488 nm and 546 nm and detection at the appropriate corresponding wavelengths (e.g. 530 nm and 590 nm, respectively). The device was an imaging fluorimeter that produces a two-dimensional electronic image of emission intensities of the array spots. A device useful for such detection is, for example, an ArrayWoRx™ microarray scanner (Applied Precision, Inc., Issaquah, Wash., USA). A detailed description of the detection process is available from the supplier (see, for example, <www.appliedprecision.com>. last visited Mar. 23, 2000). Briefly, white light is directed through an excitation filter to deliver selected monochromatic light onto to the hybridized sample. Fluorescent emission is focused on a CCD camera having high resolution capability. The collected detection data may be concurrently or subsequently analyzed and reported. Preferably, each emission color is represented separately for display purposes.

Alternative devices and procedures known in the art are useful for the detection and analysis of the relative complex formation of control and test probes with target polynucleotides according to the invention. Other useful procedures are found in, for example, WO 00/32824 (published Jun. 8, 2000), WO 00/04188 (published Jan. 27, 2000).

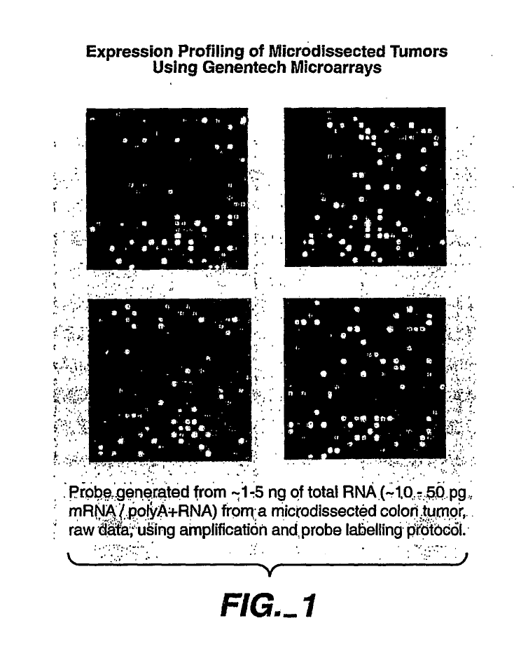

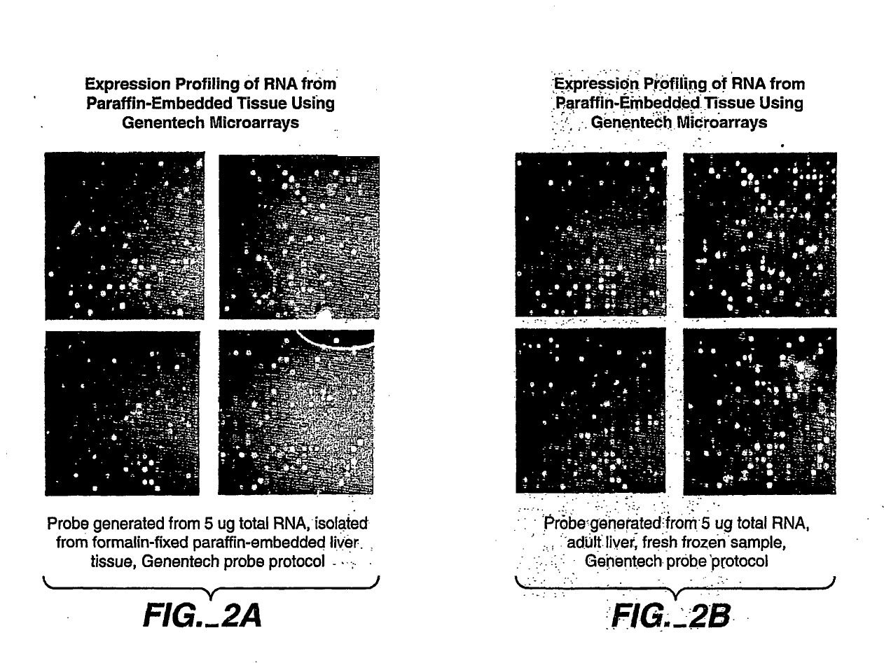

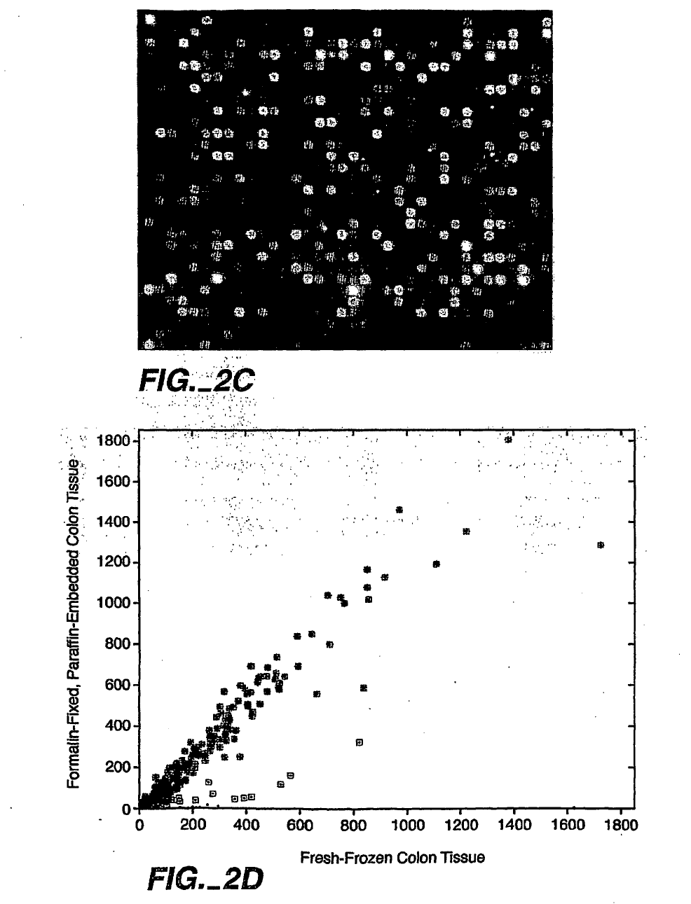

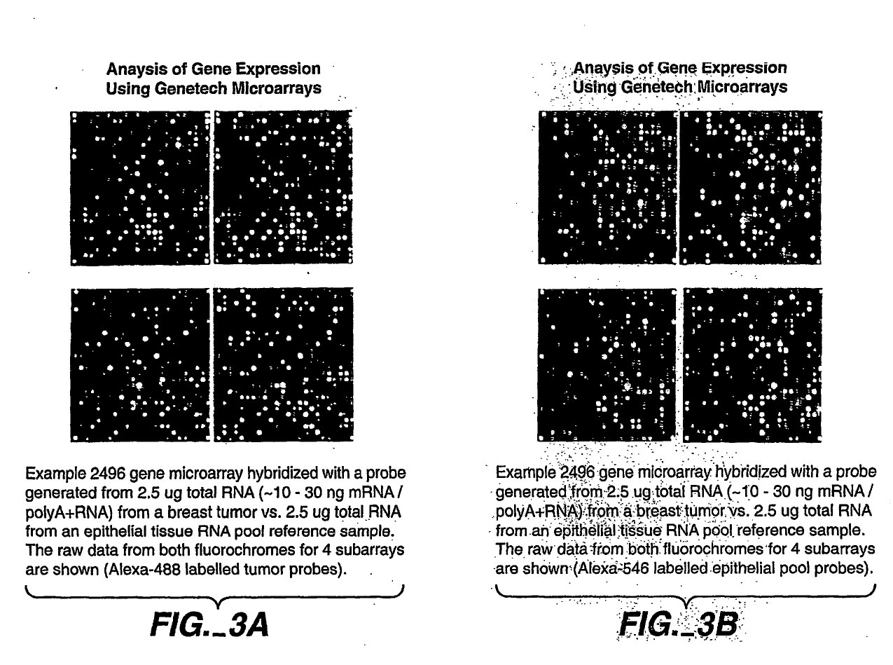

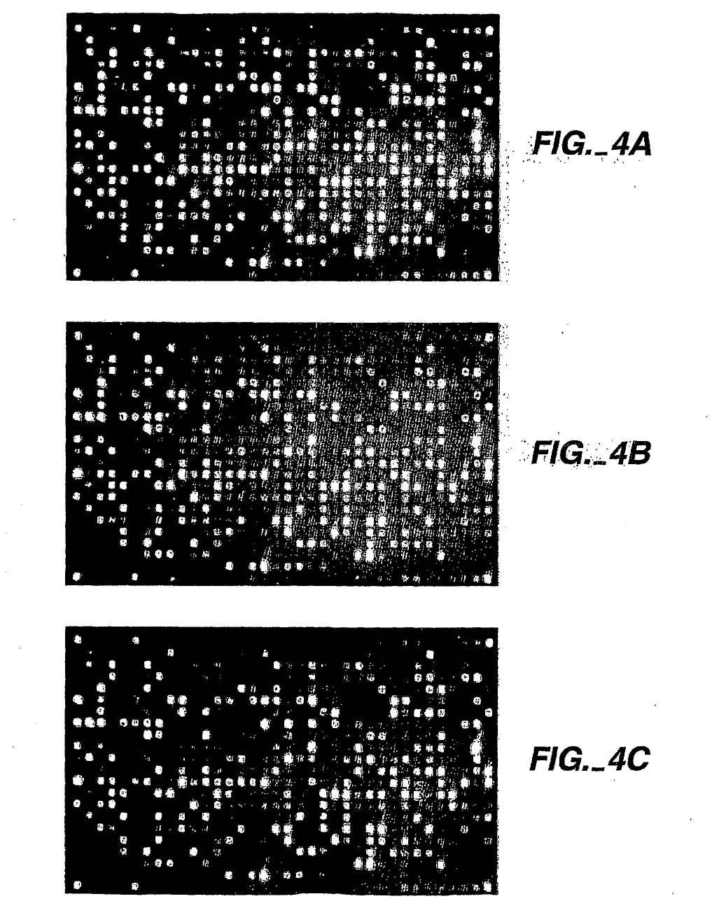

FIGS. 1-4 are examples of microarray experiment results, where the microarrays were prepared and treated according to the methods of the invention disclosed herein (i.e., RNA purification, slide preparation, probe synthesis, and probe hybridization). FIG. 1 is a photograph of microarrays hybridized with probes synthesized from a very small quantity of tumor cells microdissected from tumor tissue. The signal-to-noise is high allowing improved detection of hybridized probes. FIGS. 2A and 2B indicate that detection is comparable for probes synthesized from paraffin-embedded liver versus fresh, frozen liver. FIGS. 2C and 2D demonstrate detection of gene expression in fresh frozen versus paraffin-embedded colon tissue from the same patient. The linear clustering of the detection data from the two differently preserved tissue samples shown in the scatter plot of FIG. 2D illustrates the quantitative gene expression obtained from fresh-frozen versus formalin-fixed, paraffin-embedded tissue is very similar. FIGS. 3A and 3B show a comparison of gene expression in colon tumor relative to gene expression in the control tissue comprising pooled epithilial tissue. Emission intensity of each spot at the emission wavelengths of the chromophores are compared and analyzed to determine the actual relative gene expression in diseased and healthy tissue. FIGS. 4A-4C show that where RNA starting material from an ovarian carcinoma cell line was limited, detection of the probes hybridized to the array was possible for sDNA probes synthesized from 200 pg (FIG. 4A), 20 pg (FIG. 4B), and 2 pg (FIG. 4C) with only one round of amplification by cRNA reverse transciption to labeled sDNA in a 5-hour reaction, as disclosed herein. A 1-color analysis of fluorescence intensity is shown.

The foregoing written specification is considered sufficient to enable one skilled in the art to practice the invention. The present invention is not to be limited in scope by the examples provided since the embodiments are intended as illustrative of certain aspects of the invention and any embodiments that are functionally equivalent are within the scope of the this invention. The presentation of examples herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims. The disclosures of all citations in the specification are expressly incorporated herein by reference.

What is claimed is:

1. A microarray comprising a surface silanized with a silane in toluene in the absence of acetone or an alcohol, and a target molecule, wherein the target molecule is attached to the surface via the silane.

2. A microarray comprising a surface silanized with a silane in toluene in the absence of acetone or an alcohol, a linker, and a target molecule, wherein the target molecule is attached to the surface via the linker.

3. The microarray of claim 2, wherein the target molecule is a polynucleotide.

4. The microarray of claim 3, wherein the polynucleotide is selected from a group consisting of an oligonucleotide, DNA, amplified DNA, cDNA, single stranded DNA, double stranded DNA, PNA, RNA, and mRNA.

5. The microarray of claim 4, wherein the polynucleotide has a length in the range of about 3 bp to 10 kb.

6. The microarray of claim 5, wherein the length is in the range of about 100 bp to 5 kb.

7. The microarray of claim 6, wherein the length is in the range of about 0.3 kb to 3 kb.

8. The microarray of claim 7, wherein the length is in the range of about 0.5 kb to 2 kb.

9. The microarray of claim 4, wherein the polynucleotide is an oligonucleotide and the oligonucleotide is 25-1000 bp, 25-500, 30-200, and 50-100 bp in length.

10. The microarray or claim 2, wherein the target molecule is a polynucleotide and comprises an amine.

11. The microarray of claim 10, wherein the amine group is a primary amine.

12. The microarray of claim 11, wherein the primary amine is at the 5′ end of the polynucleotide.

13. The microarray of claim 11, wherein the primary amine is attached at the 5′ end of the polynucleotide via a linker, wherein the linker comprises one or more monomers of 1-20 carbon atoms, and wherein the monomer comprises a linear chain of carbons or a ring or both.

14. The microarray of claim 12, wherein the polynucleotide is prepared by extending a nucleic acid primer comprising a primary amine at its 5′ end.

15. The microarray of claim 2, wherein the substrate surface is selected from the group consisting of polymeric materials, glasses, ceramics, natural fibers, nylon, nitrocellulose, silicons, metals, and composites thereof.

16. The microarray of claim 15, wherein the substrate surface is planar.

17. The microarray of claim 15, wherein the substrate is in a form of threads, sheets, films, gels, membranes, beads, plates, and like structures.

18. The microarray of claim 15, wherein the substrate surface is glass.

19. The microarray of claim 18, wherein the substrate is a glass slide.

20. The microarray of claim 2, wherein the target molecule is attached after contacting the target molecule with the surface by a technique selected from the group consisting of printing, capillary device contact printing, microfluidic channel printing, deposition on a mask, and electrochemical-based printing.

21. The microarray of claim 20, wherein the target molecule is unmodified prior to the contacting.

22. The microarray of claim 21, wherein the target molecule is modified to comprise an amine prior to the contacting.

23. The microarray of claim 22, wherein the amine is a primary amine.

24. The microarray of claim 23, wherein the target molecule is a polynucleotide and the primary amine is at the 5′ end of the polynucleotide.

25. A microarray prepared by a method comprising:
(a) providing a multifunctional linker reagent comprising two or more reactive groups capable of reacting with a functional group on a surface of a microarray substrate and capable of reacting with a target molecule;
(b) activating the substrate surface for immobilizing the target molecule, by silanizing the surface with a silane in toluene in the absence of acetone or an alcohol, wherein the silane comprises a functionality reactive with the multifunctional linker reagent, and wherein the activating further comprises immobilizing the multifunctional linker reagent on the silanized surface by attaching the multifunctional linker reagent to the silane via a first reactive group of the linker reagent and a reactive group of the silane;
(c) providing a solution comprising a target molecule having one or more functional groups reactive with a second reactive group of the immobilized multifunctional linker reagent;
(d) attaching the target molecule to the substrate surface by contacting the target molecule with the activated substrate surface under conditions that promote attachment of the target molecule to the immobilized multifunctional linker reagent.

26. The microarray of claim 25, wherein the target molecule is a polynucleotide, and wherein the contacting of step (d) is carried out by spotting the polynucleotide on an activated substrate surface.

27. The microarray of claim 26, wherein the polynucleotide is unmodified.

28. The microarray of claim 26, wherein the polynucleotide is modified with an amine group.

29. The microarray of claim 28, wherein the amine group is a primary amine at the 5′ end of the polynucleotide.

30. The microarray of claim 26, wherein the polynucleotide is spotted on the surface at a concentration in the range of approximately 0.1 μg/μt to and including approximately 3 μg/μl.

31. The microarray of claim 25, wherein the attaching of step (d) occurs in a pH range from pH 6 to and including pH 10.

32. The microarray of claim 31, wherein the pH range is from pH 6.5 to and including pH 9.7.

33. The microarray of claim 32, wherein the pH range is from pH 7 to and including pH 9.4.

34. The microarray or claim 33, wherein the pH is 9.3.

35. The microarray of claim 25, wherein the attaching is allowed to occur for a time period from 1 minute to and including 24 hours.

36. The microarray of claim 35, wherein the time period is from 1-24 hours.

37. The microarray of claim 36, wherein the time period is from 5-18 hours.

38. The microarray of claim 37, wherein the time period is from 10-16 hours.

39. The microarray of claim 38, wherein the time period is from 12-14 hours.

40. The microarray of claim 25, wherein the method of preparing the microarray further comprises, after step (d), blocking unreacted reactive groups.

41. An activated slide comprising a substrate surface comprising a silane attached thereto, wherein the silanizing was in toluene, in the absence of acetone or an alcohol, and wherein the attached silane comprises at least one reactive functionality that is capable of reacting with a compound to immobilize the compound on the substrate surface.

42. The activated slide of claim 41, wherein the compound is selected from the group consisting of a modified target molecule, an unmodified target molecule, and a multifunctional linker reagent.

43. The activated slide of claim 42, wherein the compound is a multifunctional linker reagent comprising at least one reactive group capable of reacting with a target molecule to immobilize the target molecule on the substrate.

44. The activated slide of claim 42, wherein the target molecule is an unmodified polynucleotide comprising a native reactive group capable of reacting with the reactive functionality of the silane.

45. The activated slide of claim 43, wherein the target molecule is an unmodified polynucleotide comprising a native reactive group capable of reacting with the reactive group of the multifunctional linker reagent.

46. The activated slide of claim 43, wherein the target molecule is a modified polynucleotide comprising a non-native reactive group capable of reacting with the reactive group of the multifunctional linker reagent.

47. The activated slide of claim 46, wherein the target molecule is a polynucleotide and the non-native reactive group is an amine.

48. The activated slide of claim 47, wherein the amine is a primary amine.

49. The activated slide of claim 48, wherein the primary amine is at the 5′ end of the polynucleotide.

50. The activated slide of claim 41, wherein the silane is an alkyl silane and the alkyl moiety is selected from the group consisting of an ethyl-, a propyl-, a butyl-, a pentyl-, a hexyl-, a heptyl-, an octyl-, a nonyl-, and a decylalkyl moiety, and the reactive functionality of the silane is selected from the group consisting of an amine, a hydroxyl moiety, an epoxide, a thiol, and a halide, and the reactive functionality is covalently linked to the alkyl moiety.

51. The activated slide of claim 50, wherein the reactive functionality of the silane is a primary amine on the alkyl moiety, and wherein at least one reactive group of the multifunctional linker reagent is a thiocyanate moiety, and wherein the multifunctional linker reagent is immobilized by covalent reaction with the primary amine of the silane of the silanized surface.

52. A method of activating a glass slide for immobilizing a target molecule, the method comprising silanizing the slide with a silane in toluene in the absence of acetone or an alcohol, wherein the silane is an alkyl silane and the alkyl moiety is selected from the group consisting of an ethyl-, a propyl-, a butyl-, a pentyl-, a hexyl-, a heptyl-, an octyl-, a nonyl-, and a decylalkyl moiety, and the reactive functionality of the silane is selected from the group consisting of an amine, a hydroxyl moiety, an epoxide, a thiol, and a halide, and the reactive functionality is covalently linked to the alkyl moiety.

53. The method of claim 52 further comprising reacting the silane with a multifunctional linker reagent comprising at least one reactive group capable of reacting with the silane and at least one reactive group capable of reacting with the target molecule for immobilizing the target molecule, wherein the reactive functionality of the silane is a primary amine on the alkyl moiety, and wherein at least one reactive group of the multifunctional linker reagent is a thiocyanate moiety, and wherein the multifunctional linker reagent is immobilized by covalent reaction with the primary amine of the silane of the silanized surface.

54. The method of claim 52, wherein the silane is an alkyl silane and the alkyl moiety is selected from the group consisting of an ethyl-, a propyl-, a butyl-, a pentyl-, a hexyl-, a heptyl-, an octyl-, a nonyl-, and a decylalkyl moiety, and the reactive functionality of the silane is selected from the group consisting of an amine, a hydroxyl moiety, an epoxide, a thiol, and a halide, and the reactive functionality is covalently linked to the alkyl moiety.

55. A method of preparing a microarray, the method comprising:
(a) providing an activated slide comprising a substrate surface comprising a silane attached thereto, wherein the silanizing was in toluene, in the absence of acetone or an alcohol, and wherein the attached silane comprises at least one reactive functionality that is capable of reacting to immobilize a target molecule on the substrate surface;
(b) reacting the activated slide surface with the target molecule under conditions to immobilize the target molecule, wherein the target molecule is selected from the group consisting of a nucleic acid, a polynucleotide, RNA, single stranded DNA, double stranded DNA, an oligonucleotide, a peptide nucleic acid (PNA), a polypeptide, a protein, an antibody, a receptor, and a ligand.

56. The method of claim 55, further comprising after step (a) reacting the activated slide surface with a multifunctional linker reagent comprising at least two reactive groups capable of reacting with the silane to immobilize the multifunctional linker reagent on the surface, wherein the activated surface comprises the multifunctional linker reagent capable of reacting with the target molecule to immobilize the target molecule on the surface.

57. The method of claim 55, wherein the target molecule is a nucleic acid, a polynucleotide, a RNA, a single stranded DNA, a double stranded DNA, an oligonucleotide, or a peptide nucleic acid.

58. The method of claim 56, wherein the target molecule is a nucleic acid, a polynucleotide, a RNA, a single stranded DNA, a double stranded DNA, an oligonucleotide, or a peptide nucleic acid, and the multifunctional linker reagent reactive group is an isothiocyanate and the linker comprises from 1 to 20 carbon atoms.

59. The method of claim 58, wherein the multi functional linker reagent comprises a plurality of linker monomers.

60. The method of claim 55, wherein the target molecule comprises is unmodified.

61. The method of claim 56, wherein the target molecule is modified and comprises an amine.

62. The method of claim 61, wherein the amine is a primary amine at the 5′ end or the target molecule.

63. The method of claim 55, wherein the silane is 3-aminopropyltriethoxysilane.

64. The method of claim 56, wherein the multifunctional linker reagent is 1,4-phenylene diisothiocyanate.

65. A method of preparing a detectably labeled sDNA probe capable of forming a detectable complex with a target molecule immobilized on a microarray surface, the method comprising:
(a) isolating an amount or total cellular RNA from a biological sample;
(b) synthesizing a mixture of detectably labeled sDNA probes, wherein the synthesis of sDNA comprises synthesizing first strand cDNA from the isolated RNA of step (a), synthesizing second strand cDNA using Klenow fragment of DNA polymerase I and the first strand cDNA as template, synthesizing cRNA using the double stranded cDNA as template; and synthesizing sDNA using reverse transcriptase in the presence of delectably labeled deoxyribonucleotide using the cRNA as a template;
(c) isolating the labeled sDNA probes.

66. The method of claim 65, wherein the amount of total cellular RNA comprises from 0.01 to 10 pg messenger RNA.

67. The method of claim 66, wherein the amount of total cellular RNA is from 1-5 pg.

68. The method of claim 67, wherein the amount of total cellular RNA is from 0.5-2 pg.

69. The method of claim 65, wherein the synthesizing of sDNA is also in the presence of hexamer primers under conditions that cause the sDNA probes to have an average length of 0.5-2 kb.

70. A method of preparing a detectably labeled cDNA probe capable of forming a detectable complex with a target molecule immobilized on a microarray surface, the method comprising:
(a) isolating an amount of total cellular RNA from a biological sample;
(b) synthesizing a mixture of detectably labeled cDNA probes, wherein the synthesis of cDNA comprises synthesizing first strand cDNA from the isolated RNA of step (a) in the presence of detectably labeled deoxynucleotide;
(c) isolating the labeled sDNA probes.

71. A method of preparing a detectably labeled sDNA probe capable of forming a detectable complex with a target molecule immobilized on a microarray surface, the method comprising:
(a) isolating an amount of total cellular RNA from a biological sample;
(b) synthesizing a mixture of detectably labeled sDNA probes, wherein the synthesis of sDNA comprises synthesizing a biotin-attached first strand cDNA from the isolated RNA of step (a); synthesizing second strand DNA (sDNA) using Klenow fragment of DNA polymerase I and the first strand cDNA as template in the presence of detectably labeled deoxynucleotides:
(c) contacting the biotin-attached first strand cDNA with streptavidin and removing the biotin-first strand cDNA/streptavidin complex from the labeled sDNA; and
(c) isolating the labeled sDNA probes.

72. A method of preparing a detectably labeled cRNA probe capable of forming a detectable complex with a target molecule immobilized on a microarray surface, the method comprising:
(a) isolating an amount of total cellular RNA from a biological sample;
(b) synthesizing a mixture of delectably labeled cRNA probes, wherein the synthesis or cRNA comprises synthesizing first strand cDNA from the isolated RNA of step (a), synthesizing second strand cDNA using Klenow fragment of DNA polymerase I and the first strand cDNA as template, synthesizing cRNA using the double stranded cDNA as template in the presence of detectably labeled ribonucleotides; and
(c) isolating the labeled sDNA probes.

73. The method of claim 72, further comprising after step (c) degrading the cRNA probe with RNase under conditions such that the average length of the cRNA probe is adjusted to be from 0.5 kb to 3 kb.

74. The method of claim 71, wherein the step of synthesizing second strand DNA is in the presence of hexamer primers under conditions such that the average length of the labeled sDNA probe is from approximately 0.5 kb to approximately 2 kb.

75. The method of claim 70, further comprising after step (b) decreasing the average length of the labeled cDNA probes to be from 0.5 kb to 2 kb.

76. The method of claim 75, wherein the decreasing is by limited DNase digestion.

77. The method of claim 65, wherein the biological sample is selected from the group consisting of a cell, a tissue sample, a body fluid sample, and a mixture of synthetic oligonucleotides.

78. The method of claim 65, wherein the amount of total cellular RNA is from 0.5 pg to and including 10 mg.

79. The method of claim 78, wherein the amount of total cellular RNA is from 1 pg to and including 10 μg.

80. The method of claim 79, wherein the amount of total cellular RNA is from 1 pg to and including 100 ng.

81. The method of claim 80, wherein the amount of total cellular RNA is from 1 pg to and including 10 ng.

82. The method of claim 65, wherein the detectably labeled deoxynucleotide is labeled dUTP and the synthesizing in the presence of labeled dUTP is in the absence of unlabeled dTTP.

83. The method of claim 65, wherein the detectable label is a fluorochromophore.

84. A method of analyzing a target molecule attached to a microarray, the method comprising:
(a) providing a microarray according to claim 1;
(b) contacting the attached target molecule with an agent capable of forming a detectable complex with the target molecule under conditions that allow-formation of a detectable complex;
(c) detecting formation of a detectable complex;
(d) determining the amount of a detectable complex formed.

85. The method of claim 84, wherein the agent capable of forming a detectable complex comprises:
a control mixture of sDNA probes comprising a first detectable label, wherein the probes are prepared from total cellular RNA isolated from a control sample, and
a test mixture of sDNA probes comprising a second detectable label, wherein the probes are prepared from total cellular RNA isolated from a test sample,
wherein the first and second detectable labels are distinguishable, wherein the method further comprises:
(1) pooling the control sDNA probes and the test sDNA probes;
(2) performing steps (a)-(d) of claim 84; and
(3) comparing the amount of detectable complex formed between the target molecule and the control probes relative to the amount of complex formed between the target molecule and the test probes.

86. The method of claim 84, wherein the label is optically detectable.

87. The method of claim 86, wherein the label is fluorescent.

88. The method of claim 84, wherein the contacting of step (b) occurs in the absence of detergent.

89. The method of claim 88, wherein the contacting of step (b) occurs in the presence of formamide and a one or more of dimethylsulfoxide (DMSO), tetramethylammonium chloride (TMACl), and tetraethylammonium chloride (TEACl).

90. The method of claim 89, wherein the contacting of step (b) occurs in the presence of formamide, DMSO and TMACl or TEACl, wherein the sum of the proportions of formamide and DMSO does not exceed 50%.

91. The method of claim 90, wherein the sum of the proportions of formamide and DMSO does not exceed 25%.

92. The method of claim 88, further comprising a wash step subsequent to the contacting step wherein the wash solution comprises detergent.

93. A method of hybridizing a detectable polynucleotide probe to a target polynucleotide on a support surface, the method comprising:
(a) contacting the probe with denatured target polynucleotide on the support surface in a hybridization solution comprising DMSO or formamide or both, and in the absence of detergent; and
(b) detecting formation of a complex between the target polynucleotide and the detectably labeled polynucleotide probe.

94. The method of claim 93, wherein the sum of the proportions of DMSO and formamide does not exceed 50%, and wherein the hybridization solution further comprises TMACl or TMECl or both.

95. The method of claim 94, wherein the sum of the proportions of DMSO and formamide does not exceed 25%, and wherein the hybridization solution further comprises TMACl or TMECl or both.

96. The method of claim 85, wherein the control sample comprises cells removed from a cell source by laser capture microdissection, wherein the cell source is selected from the group consisting of untreated tissue, frozen tissue, paraffin-embedded tissue, stained tissue, and cell culture.

97. The method of claim 85, wherein the test sample comprises cells removed from a cell source by laser capture microdissection, wherein the cell source is selected from the group consisting of untreated tissue, frozen tissue, paraffin-embedded tissue, stained tissue, and cell culture.

98. The method of claim 85, wherein the test sample and control sample differ according to one or more of developmental state, disease state, pre-disease state, cell type, sample source, and experimental treatment conditions.

99. The method of claim 85, wherein the target molecule is a polynucleotide and the nucleic acid isolated from the test sample and the control sample is RNA, and wherein the comparing of step (c) provides a measure of target polynucleotide expression in the test sample relative to target polynucleotide expression in the control sample.

100. The method of claim 99, wherein the relative measure of target polynucleotide expression indicates a disease state in the test tissue sample.

101. The method of claim 100, wherein the disease state is selected from the group consisting of tumor, cardiovasular disease, inflammatory disease, endocrine disease.

102. The method of claim 100, wherein the relative measure of target polynucleotide expression indicates a pre-disease state in the test tissue sample.

103. The method of claim 84, wherein the target molecule is a polynucleotide and the nucleic acid isolated from the test sample and the control sample is DNA, and wherein the comparing of step (c) provides a measure of number of copies of the target polynucleotide in cells of the test sample relative to target polynucleotide copies in the control sample.

104. The method of claim 103, wherein the relative measure of the number of copies of target polynucleotide indicates a disease state or a pre-disease state in the test tissue sample.
